SEQUENCE LISTING (24 sequences)

SEQ ID NO: 1 - protein, 1273 aa, Severe acute respiratory syndrome-related coronavirus
  source 1..1273: note = SARS-CoV-2 spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 12 aa, Severe acute respiratory syndrome-related coronavirus
  source 1..12: note = SARS-CoV-2 S protein signal peptide
MFVFLVLLPLVS

SEQ ID NO: 3 - protein, 673 aa, Severe acute respiratory syndrome-related coronavirus
  source 1..673: note = SARS-CoV-2 S protein S1 subunit
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 4 - protein, 588 aa, Severe acute respiratory syndrome-related coronavirus
  source 1..588: note = SARS-CoV-2 S protein S2 subunit
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 223 aa, Severe acute respiratory syndrome-related coronavirus
  source 1..223: note = SARS-CoV-2 S protein receptor binding domain (RBD)
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 6 - protein, 222 aa, Severe acute respiratory syndrome-related coronavirus
  source 1..222: note = SARS-CoV-2 membrane protein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 7 - protein, 75 aa, Severe acute respiratory syndrome-related coronavirus
  source 1..75: note = SARS-CoV-2 envelope protein
MYSFVSEETGTLIVNSVLLFLAFVVFLLVTLAILTALRLCAYCCNIVNVSLVKPSFYVYSRVKNLNSSRVPDLLV

SEQ ID NO: 8 - protein, 419 aa, Severe acute respiratory syndrome-related coronavirus
  source 1..419: note = SARS-CoV-2 nucleoprotein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 9 - protein, 18 aa, Measles morbillivirus
  source 1..18: note = Measles Virus Fusion Protein
KLLSLIKGVIVHRLEGVE

SEQ ID NO: 10 - protein, 19 aa, Clostridium tetani
  source 1..19: note = Tetanus Toxin
NSVDDALINSTIYSYFPSV

SEQ ID NO: 11 - protein, 17 aa, Clostridium tetani
  source 1..17: note = Tetanus Toxin
PGINGKAIHLVNNQSSE

SEQ ID NO: 12 - protein, 15 aa, Clostridium tetani
  source 1..15: note = Tetanus Toxin P2
QYIKANSKFIGITEL

SEQ ID NO: 13 - protein, 21 aa, Clostridium tetani
  source 1..21: note = Tetanus Toxin P30
FNNFTVSFWLRVPKVSASHLE

SEQ ID NO: 14 - protein, 15 aa, Measles morbillivirus
  source 1..15: note = Measles Virus Fusion Protein
LSEIKGVIVHRLEGV

SEQ ID NO: 15 - protein, 15 aa, Hepatitis B virus
FFLLTRILTIPQSLN

SEQ ID NO: 16 - protein, 21 aa, Plasmodium sp.
  source 1..21: note = circumsporozoite protein
TCGVGVRVRSRVNAANKKPEL

SEQ ID NO: 17 - protein, 21 aa, Hepacivirus C
  source 1..21: note = Core-E1 signal peptidase - HCV genotype 1a
GCSFSIFLLALLSCLTVPASA

SEQ ID NO: 18 - protein, 21 aa, Hepacivirus C
  source 1..21: note = Core-E1 signal peptidase - HCV genotype 1b
GCSFSIFLLALLSCLTIPASA

SEQ ID NO: 19 - protein, 21 aa, Hepacivirus C
  source 1..21: note = Core-E1 signal peptidase - HCV genotype 2a
GFPFSIFLLALLSCITVPVSA

SEQ ID NO: 20 - protein, 21 aa, Hepacivirus C
  source 1..21: note = Core-E1 signal peptidase - HCV genotype 3a
GCSFSVFLLALFSCLIHPAAS

SEQ ID NO: 21 - protein, 21 aa, Hepacivirus C
  source 1..21: note = Core-E1 signal peptidase - HCV genotype 4a
GCSFSIFLLALLSCLTVPASA

SEQ ID NO: 22 - protein, 21 aa, Hepacivirus C
  source 1..21: note = Core-E1 signal peptidase - HCV genotype 5a
GCSFSIFILALLSCLTVPTSA

SEQ ID NO: 23 - protein, 21 aa, Hepacivirus C
  source 1..21: note = Core-E1 signal peptidase - HCV genotype 6a
GCSFSIFLLALLSCLTTPASA

SEQ ID NO: 24 - protein, 21 aa, Hepacivirus C
  source 1..21: note = Core-E1 signal peptidase - HCV genotype 7a
GCSFSIFLLALLSCLTVPASA

(19) United States
(12) Patent Application Publication
(10) Pub. No.: US 2025/0186575 A1
(43) Pub. Date: Jun. 12, 2025

(54) VACCINE CONSTRUCT AND USES THEREOF

(71) Applicant: The University of Melbourne, Victoria (AU)

(72) Inventor: Joseph Torresi, Victoria (AU)

(73) Assignee: The University of Melbourne, Victoria (AU)

(21) Appl. No.: 18/294,857

(22) PCT Filed: Aug. 4, 2022
(86) PCT No.: PCT/AU2022/050843

§ 371 Date: Feb. 2, 2024

(30) Foreign Application Priority Data
Aug. 4, 2021 (AU) 2021902407

(51) Int. Cl. A61K 39/215; A61K 39/00; A61P 31/14; C07K 14/005; C12N 7/00

(52) U.S. Cl. CPC A61K 39/215; A61P 31/14; C07K 14/005; C12N 7/00; A61K 2039/55572; C12N 2770/20022; C12N 2770/20023; C12N 2770/20034; C12N 2770/20051

(57) ABSTRACT

Disclosed herein are nucleic acid constructs for producing a virus-like particle (VLP) capable of raising an immune response against severe acute respiratory syndrome coronavirus (SARS-CoV), and uses thereof, wherein the constructs comprise nucleic acid sequences encoding an immunogen and a polyprotein, wherein the polyprotein comprises two or more viral structural proteins, wherein at least two of the two or more viral structural proteins are separated by a signal peptidase sequence such that, when the polyprotein is expressed in a host cell, the signal peptidase sequence undergoes host cell peptidase-dependent cleavage to liberate the two or more viral structural proteins, thereby allowing the liberated structural proteins to self-assemble into a VLP carrying the immunogen.

### FIELD OF THE INVENTION

The present invention relates generally to a nucleic acid construct for producing a virus-like particle capable of raising an immune response against severe acute respiratory syndrome coronavirus, and uses thereof.

### BACKGROUND

The World Health Organisation (WHO) reports that the severe acute respiratory coronavirus 2 (SARS-CoV-2) pandemic that was first reported in 2019 has, as of 24 Mar. 2021, infected more than 124 million people and caused almost 3 million deaths. The infectious disease caused by SARS-CoV-2 is often referred to as COVID-19. Most people infected with SARS-CoV-2 will experience mild to moderate respiratory illness and recover without requiring special treatment. However, reports show that around 12-18% of COVID-19 patients will develop severe disease requiring hospitalization, around 5% are critical, while others who are less severely affected, and even asymptomatic, may still have some underlying pathology. Older people, and those with underlying medical problems like cardiovascular disease, diabetes, chronic respiratory disease, and cancer are more likely to develop serious illness.

There are a myriad of SARS-CoV-2 vaccines in development. Whilst some vaccines having already received approval from regulatory authorities, it is becoming clear that these vaccines vary significantly in efficacy, including their ability to prevent severe illness. Moreover, as variants of SARS-CoV-2 continue to emerge globally (e.g., UK variant 20I/501Y.V1, also known as VOC 202012/01 or B.1.1.7; South Africa variant 20H/501Y.V2, also known as B.1.351; and Brazil variant P.1), there is growing doubt as to whether existing SARS-CoV-2 vaccines will be sufficient to provide protection against infection by new strains of the virus, and especially those that carry a E484K mutation in the receptor binding motif (RBM).

Thus, there remains a critical need for improved vaccine compositions capable of inducing protective immunity against SARS-CoV, and in particular against SARS-CoV-2.

### SUMMARY

In an aspect disclosed herein, there is provided a nucleic acid construct for producing a virus-like particle (VLP) capable of raising an immune response against severe acute respiratory syndrome coronavirus (SARS-CoV), wherein the construct comprises a nucleic acid sequence encoding a polyprotein, wherein the polyprotein comprises (i) an immunogen; and (ii) two or more viral structural proteins, wherein the immunogen comprises a B cell epitope and/or a T cell epitope of a SARS-CoV surface protein and wherein at least two of the two or more viral structural proteins are separated by a signal peptidase sequence such that, when the polyprotein is expressed in a host cell, the signal peptidase sequence undergoes host cell peptidase-dependent cleavage to liberate the two or more viral structural proteins, thereby allowing the liberated structural proteins to self-assemble into a VLP carrying the immunogen.

In another aspect disclosed herein, there is provided a method of producing a VLP, the method comprising:
(i) introducing the nucleic acid construct described herein into a host cell; and
(ii) culturing the host cell of (i) under conditions and for a period of time sufficient for the host cell to produce the VLP.

The present disclosure also extends to a system or composition comprising (i) a first construct comprising a nucleic acid sequence encoding an immunogen, wherein the immunogen comprises a B cell epitope and/or a T cell epitope of a SARS-CoV surface protein; and (ii) a second construct comprising a nucleic acid sequence encoding a polyprotein, wherein the polyprotein comprises two or more viral structural proteins, wherein at least two of the two or more viral structural proteins are separated by a signal peptidase sequence such that, when the polyprotein and the immunogen are expressed in a host cell, the signal peptidase sequence of the polyprotein undergoes host cell peptidase-dependent cleavage to liberate the two or more viral structural proteins, thereby allowing the liberated structural proteins and the immunogen to self-assemble into a VLP.

In another aspect disclosed herein, there is provided a method of producing a VLP, the method comprising:
(i) introducing the first and second vaccine constructs, as described herein, into a host cell; and
(ii) culturing the host cell of (i) under conditions and for a period of time sufficient for the host cell to produce the VLP.

The present disclosure also extends to a VLP produced by the methods described herein and a vaccine composition comprising the nucleic acid construct or the VLP, described herein.

In another aspect disclosed herein, there is provided a method of raising an immune response in a subject to a SARS-CoV, the method comprising administering to a subject in need thereof the nucleic acid construct, the VLP or the composition, as described herein.

In another aspect disclosed herein, there is provided use of the nucleic acid construct, the VLP or the composition, as described herein, in the manufacture of a medicament for raising an immune response in a subject to a SARS-CoV.

In another aspect disclosed herein, there is provided the nucleic acid construct, the VLP or the composition, as described herein, for use in raising an immune response in a subject against a SARS-CoV.

Also disclosed herein is a kit comprising the nucleic acid construct, the VLP and/or the system or compositions, as described herein.

In another aspect disclosed herein, there is provided a host cell comprising the nucleic acid constructs as described herein.

### BRIEF DESCRIPTION OF THE FIGURES

FIG. 1 shows a map of the genes encoding the SARS-CoV-2 spike protein and the receptor binding domain (RBD). Also depicted and described are known mutations associated with SARS-CoV2 variants of concern.

FIG. 2 shows a polyprotein map of the SARS-CoV-2 vaccine construct with signal peptide peptidase (SPP) sequences utilized by HCV to facilitate host cell dependent-peptidase cleavage and release of the monomeric proteins for self-assembly into VLP. The receptor binding domain (RBD) at amino acid positions 415-492 of the C-terminal end of the S1 protein is shown. The D614G mutation in the C terminal end of the S1 protein and two stabilizing proline substitutions (K986P and V987P) are also shown. The interchangeable region in the S protein encompassing S1 and the RBD to introduce new sequences of emerging variants of concern using Gibson Isothermal Assembly is indicated below the map.

FIG. 3 shows the stepwise production of recombinant adeno-SARS-CoV-2 polyprotein in HEK 293T cells (Steps 1-4) and the subsequent production of SARS-CoV-2 VLP Vero cells, as determined by fluorescence microscopy (SARS-CoV-2 SEM VLP refers to a SARS-CoV-2 VLP comprising the structural (S), envelope (E) and the membrane (M) proteins).

FIG. 4 shows the presence of recombinant SARS-CoV-2 SEM VLP and SARS-CoV-2 virus following infection of Vero cells, as determined by Western blot analysis. (A) Immunoblot of SARS-CoV2 SEM VLP probed with acute convalescent IgG purified from serum of a patient who recovered from COVID-19 infection. (B) Western blot of SARS-CoV2 SEM VLP and SARS-CoV2 virus probed with polyclonal anti-S1/RBD antibody (MyBioSource). (C) Immunoblot probed with immune IgG from an individual who had been infected with 229E coronavirus showing no reactivity to the SARS-CoV-2 VLP or to the SARS-CoV-2 virus; (D) similar immunoblot probed with IgG from pre-pandemic healthy controls showing no reactivity to the SARS CoV2 VLP or to the SARS-CoV-2 virus.

FIG. 5 shows (A) Western blot confirming the presence of the S protein in the SARS CoV-2 VLP. The blot was probed with a rabbit polyclonal anti-S1 antibody (SinoBiological). (B) Atomic force microscopy (AFM) of purified SARS-CoV2 VLPs (C) Transmission electron microscopy of negatively stained SARS CoV-2 VLP showing typical pleomorphic 50 to 100 nm particles with characteristic spikes. Immunogold EM (bottom right) showing gold beads bound to the RBD of spikes with an anti-SARS CoV-2 RBD antibody (MyBioSource).

FIG. 6 shows ELISA results using an anti-SARS-CoV-2 RBD antibody (MyBioSource) to confirm the presence of the RBD on SARS-CoV-2 SEM VLP coated onto the ELISA plates. The plates were coated with increasing concentrations of SARS CoV2 VLP and then probed with increasing dilutions of the anti-RBD antibody. The results show that the anti-RBD antibody recognizes the RBD present on native, non-denatured SARS-CoV-2 VLP.

FIG. 7 shows the immunogenicity of the SARS-CoV-2 VLP in mice following administration of two doses of vaccine 7 days apart and harvesting at day 10, as determined by (i) ELISA for the presence of an anti-S1 (A) and anti-RBD antibodies (B), (C) B cell ELISPOT enumerating antibody secreting cells in different groups of immunized mice, and (D) flow cytometry for CD19-negative NKT cells in liver of immunized mice. The results were compared to the administration of the SARS-CoV-2 VLP in the presence of alpha-Galactosylceramide (α-GalCer) or the squalene-based, oil-in-water nano-emulsion vaccine adjuvant, AddaVax (Invivogen).

FIG. 8 shows the immunogenicity of the SARS-CoV-2 VLP in mice following administration of two doses of vaccine 14 days apart and harvesting at day 21, as determined by ELISA for the presence of (A) anti-S1 antibody with individual immune mouse sera (from left to right: PBS pre-bleed; PBS day 14; PBS day 21; SARS-CoV2 VLP pre-bleed; SARS-CoV2 VLP day 14; SARS-CoV2 VLP day 21; α-GC-SARS-CoV2 VLP pre-bleed; α-GC-SARS-CoV2 VLP day 14; α-GC-SARS-CoV2 VLP day 21; AddaVax/SARS-CoV2 VLP pre-bleed; AddaVax/SARS-CoV2 VLP day 14; AddaVax/SARS-CoV2 VLP day 21), (B) anti-RBD antibodies (from left to right: SARS-CoV2 VLP; α-GC-SARS-CoV2 VLP; AddaVax/SARS-CoV2 VLP; PBS) and (C) Durability of antibody responses following administration of three doses of vaccine on days 0, 14 and 28 and harvesting at day 70 determined by ELISA. The results were compared to the administration of the SARS-CoV-2 VLP in the presence of α-GC or the squalene-based, oil-in-water nano-emulsion vaccine adjuvant, AddaVax (Invivogen).

FIG. 9 shows the immunogenicity of the SARS-CoV-2 VLP in mice following administration of two doses of vaccine 14 days apart and harvesting at day 21. T-cell responses were determined by stimulating splenocytes for 4 days with SARS-CoV-2_{SEM} VLP before plating onto anti-γ-IFN coated ELISPOT plates. Mice in all vaccine groups developed very strong T cell responses. Mice vaccinated with SARS-CoV-2_{SEM} VLP/Addavax developed the strongest responses (7763+/−SD 142 SARS-CoV-2_{SEM} specific γ-IFN secreting cells/million splenocytes). Mice vaccinated with α-GalCer-SARS-CoV-2_{SEM} VLPs also developed strong T cell responses (7180+/−SD 95 SARS-CoV-2_{SEM} specific γ-IFN secreting cells/million splenocytes) demonstrating that the self-adjuvanted vaccine is also highly effective. Encouragingly, mice receiving SARS-CoV-2_{SEM} VLP alone also developed strong T cell responses (6591+/−SD 42 SARS-CoV-2_{SEM} specific γ-IFN secreting cells/million splenocytes), although less than the adjuvanted vaccines.

FIG. 10 shows the immunogenicity of the SARS-CoV-2 VLP in mice following administration of two doses of vaccine 14 days apart and harvesting at day 21. B cell responses to the vaccines were determined by stimulating splenocytes with R848 and IL2 before adding cells to SARS-CoV-2_{SEM} VLP coated B cell ELISPOT plates. Strong SARS-CoV-2_{SEM} specific B cell responses were detected in all vaccine groups with the strongest responses again in mice vaccinated with SARS-CoV-2_{SEM} VLP/Addavax vaccine (439+/−16 SARS-CoV-2_{SEM} specific ASC/million splenocytes) compared with α-GalCer-SARS-CoV-2_{SEM} VLP (338+/−63 SARS-CoV-2_{SEM} specific ASC/million splenocytes) and SARS-CoV-2_{SEM} VLP alone (296+/−15 SARS-CoV-2_{SEM} specific ASC/million splenocytes).

FIG. 11 shows (A) the presence of neutralizing anti-SARS-CoV-2 antibodies in mice at following administration of two doses of phosphate buffered saline (PBS; controls) or the SARS-COV-2 VLP with adjuvant (Addavax™) by intramuscular injection (2 weeks apart). Blood samples were taken at 2 weeks (M1), 4 weeks (M2) and 6 weeks (M3) after the second (booster) dose. The presence of neutralizing anti-SARS-CoV-2 antibodies was determined a SARS-CoV-2 Surrogate Virus Neutralization Test (sVNT; GenScript) and the results are expressed as the % inhibition of the interaction between the receptor binding domain (RBD) of the SARS-CoV-2 spike glycoprotein and the angiotensin converting enzyme 2 (ACE2) human cell surface receptor. Positive control is a potent neutralizing monoclonal antibody. The negative control is PBS; (B) the inhibition of SARS-CoV-2 infection in mice who were challenged with infectious SARS-CoV-2 virus particles at 8 weeks since the first dose (6 weeks after the second, booster dose). The results are expressed as the number of SARS-CoV-2 virus particles (Log SARS-CoV2 TCID₅₀) per lung; and (C) the number of IFNγ-secreting T cells in the splenocytes collected from these mice. The results are expressed as the number of IFNγ-secreting T cells per 10⁶ splenocytes.

FIG. 12 shows the inhibition of SARS-CoV-2_{VIC01} infection of human nasal epithelial cells in the presence of sera collected from mice that received PBS alone (PBS controls) or from mice immunized with the ancestral SARS-CoV-2 VLP plus Addavax™ (VLP/Ad). The data, expressed as the Median Tissue Culture Infectious Dose (TCID₅₀), were compared to uninfected human nasal epithelial cells and with human nasal epithelial cells infected with VIC01 SARS-CoV-2 in the presence of a known neutralizing antibody. The positive control used was a known potent SARS-CoV-2 neutralizing antibody.

FIG. 13 shows that immunization of mice produces a predominant CD4-T cell response. Splenocytes were harvested from mice immunized with SARS-CoV-2 vaccine alone or combined with Addavax™ followed by depletion of CD4+ or CD8+ T cells and analysis by Elispot assays. Depletion of CD4+ T cells resulted in a significant reduction in the number of spots in both the VLP alone and VLP/Addavax™ vaccinated mice. The results are shown for categories (i) non-depleted, (ii) CD4 depleted and (iii) CD8 depleted T cells; from left to right for each category: PBS, VLP and VLP+_Addavax™

FIG. 14 shows that purified ß-SARS-CoV-2 VLP produced by Vero cells express the receptor-binding domain (RBD), as determined by ELISA. Plates were coated with either ß-SARS-CoV-2 VLPs or a ß-RBD positive control antigen. The plates were probed with a SARS-CoV-2 anti-S antibody (MyBioSource; MBS2563840). The reactivity of the ß-SARS-CoV-2 VLPs was comparable to ß-RBD positive control antigen.

FIG. 15 shows that purified ß-SARS-CoV-2 VLP produced by Vero cells express the SARS-CoV-2 spike protein, as determined by ELISA. Plates were coated with either ß-SARS-CoV-2 VLP or a ß-RBD positive control antigen. The plates were then probed with a SARS-CoV-2 anti-S antibody (SinoBiologic; 40491-T62). The reactivity of the ß-SARS-CoV-2 VLP was comparable to ß-RBD positive control antigen.

FIG. 16 shows a Western immunoblot of purified ß-SARS-CoV-2 VLPs produced from cell factories. The VLP were separated by SDS-PAGE and transferred to PVDF membrane. The blot was probed with an anti-spike antibody (MyBioSource; MBS2563840). A strong band as detected at approximately 105 kDa in lanes 4 and 5 consistent with the presence of the spike protein. The positive control is a recombinant spike protein. The negative control is PBS.

FIG. 17 shows the formation of ßSARS-CoV-2 VLPs by Vero cells, as determined by transmission electron microscope (TEM).

FIG. 18 shows the presence of the spike protein (S; FIG. 18A), the membrane protein (M; FIG. 18B) and the envelope protein (E; FIG. 18C) on the ßSARS-CoV-2 VLP produced by Vero cells carrying the dual constructs encoding the ßSARS-CoV-2 VLP.

FIG. 19 shows an updated map of the genes encoding the SARS-CoV-2 spike protein and the receptor binding domain (RBD). Also depicted and described are known mutations associated with SARS-CoV2 variants of concern.

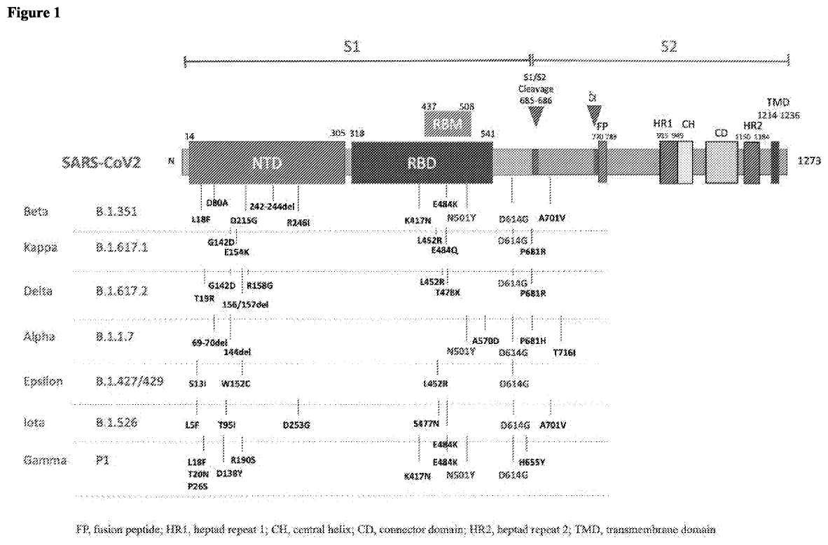

### DETAILED DESCRIPTION

Unless defined otherwise, all technical and scientific terms used herein have the same meanings as commonly understood by one of ordinary skill in the art to which the present disclosure belongs. Any materials and methods similar or equivalent to those described herein can be used to practice the present invention. Practitioners may refer to Sambrook et al., (1989) Molecular Cloning: A Laboratory Manual, 2nd ed., Cold Spring Harbor Press, Plainsview, N.Y., and Ausubel et al. (1999) Current Protocols in Molecular Biology (Supplement 47), John Wiley & Sons, New York, Murphy et al. (1995) Virus Taxonomy Springer Verlag:79-87, for definitions and terms of the art and other methods known to the person skilled in the art.

The disclosure of every patent, patent application, and publication cited herein is hereby incorporated herein by reference in its entirety.

The citation of any reference herein should not be construed as an admission that such reference is available as “Prior Art” to the instant application.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein the singular forms “a”, “an” and “the” include plural aspects unless the context clearly dictates otherwise. Thus, for example, reference to “a cell” includes a single cell, as well as two or more cells; reference to “a VLP” includes one VLP, as well as two or more VLP; and so forth.

As used herein, the term “about” refers to approximately a +/−10% variation from a given value. It is to be understood that such a variation is always included in any given value provided herein, whether or not it is specifically referred to.

The present invention is predicated, at least in part, on the inventors' surprising finding that a nucleic acid construct encoding a self-cleaving polyprotein comprising SARS-CoV structural proteins is capable of self-assembling into stable virus-like particles (VLP) that are capable of generating an immune response against SARS-CoV. Thus, in an aspect disclosed herein, there is provided a nucleic acid construct for producing a virus-like particle (VLP) capable of raising an immune response against severe acute respiratory syndrome coronavirus (SARS-CoV), wherein the construct comprises a nucleic acid sequence encoding a polyprotein, wherein the polyprotein comprises (i) an immunogen; and (ii) two or more viral structural proteins, wherein the immunogen comprises a B cell epitope and/or a T cell epitope of a SARS-CoV surface protein and wherein at least two of the two or more viral structural proteins are separated by a signal peptidase sequence such that, when the polyprotein is expressed in a host cell, the signal peptidase sequence undergoes host cell peptidase-dependent cleavage to liberate the two or more viral structural proteins, thereby allowing the liberated structural proteins to self-assemble into a VLP carrying the immunogen.

### Virus-Like Particles

Virus-like particles (VLP) are typically defined as self-assembled complexes of viral structural proteins (e.g., capsid and/or envelope proteins) that mimic the structure of their parental virus, however, as none of the replicating nucleic acids of the parental virus are present, the VLP cannot replicate virus genome and create more or otherwise infectious virus particles. VLP are strictly non-infectious and generally harmless to the environment. When VLP are formed in the presence of an immunogen, the VLP can suitably be used as a delivery vehicle for the immunogen. VLP can also possess an antigenicity similar to the parent virus from which the structural components were obtained or derived and are therefore useful as vaccines against that particular virus infection.

As noted above, VLP will typically lack, or possess dysfunctional copies of, certain genes of the native virus. As a consequence, VLP are generally incapable of some function that is otherwise characteristic of the native or parental virus, such as replication and/or cell-cell movement. Typically void of viral genetic material, VLP possess biologically desirable traits that are attributed, at least in part, to the particulate viral structure. Of particular interest is their efficient recognition, cellular uptake, and processing by host immune systems. VLP are also amenable to a broad range of modifications including encapsulation, chemical conjugation, and genetic manipulation (see, e.g., Roldio et al. Expert Rev Vaccines 2010; 9(10):1149-76). This versatility of VLP has prompted their use as suitable delivery agents for immunotherapy, noting that licensed prophylactic VLP vaccines such as Gardasil®, Cervarix®, Hecolin®, and Porcilis PCV® highlight VLP vaccines as being safe and effective. VLP also overcome some of the drawbacks associated with traditional vaccine production; namely, the infectious nature associated with live and inactivated vaccines and lengthy production time.

The term “self-assembly” typically refers to a process in which a system of pre-existing components, under specific conditions, adopts a more organised structure through interactions between the components themselves. In the context of VLP, self-assembly typically refers to the intrinsic capacity of the viral structural proteins to self-assemble into a VLP when subjected to conditions that support such assembly. It is to be understood that self-assembly does not preclude the role of other cellular proteins such as chaperons in the process of VLP assembly. Thus, viral structural proteins may be able to self-assemble and form VLP on their own, or in combination with several virus proteins. The self-assembly process may be influenced by factors known to persons skilled in the art, such as the choice of expression host, the choice of expression conditions, and the conditions for maturing the VLP. The terms “virus-like particle” and “VLP” are therefore used interchangeably herein to refer to macromolecular particulate structures, formed by the self-assembly of recombinantly expressed viral structural proteins, that mimick the morphology of a virus coat, but otherwise lack infectious genetic material.

### Immunogens

The term “immunogen”, as used herein, typically refers to a molecule, molecules, a portion or portions thereof, or a combination of molecules, which are capable of inducing an immune response in a subject. The immunogen may suitably comprise a single epitope or it may comprise a plurality of epitopes, including B cell and T cell epitopes or mimotopes thereof. Immunogens may therefore encompass peptides, proteins, carbohydrates, and nucleic acids.

An immunogen is typically capable of raising an immune response, including a humoral (antibody) and/or cellular immune response, in vivo, whether alone or when combined (e.g., co-administered to a subject) with a suitable adjuvant. The terms “peptide” and “polypeptide” are used interchangeably herein in their broadest sense to refer to a molecule of two or more amino acid residues, or amino acid analogs. The amino acid residues may be linked by peptide bonds, or alternatively by other bonds, e.g. ester, ether etc., but in most cases will be linked by peptide bonds. The terms “amino acid” or “amino acid residue” are used herein to encompass both natural and unnatural or synthetic amino acids, including both the D- or L-forms, and amino acid analogs. An “amino acid analog” is to be understood as a non-naturally occurring amino acid differing from its corresponding naturally occurring amino acid at one or more atoms. For example, an amino acid analog of cysteine may be homocysteine.

The polyprotein encoded by the nucleic acid construct described herein will suitably comprise at least one immunogen. By “at least one immunogen” is meant 1, 2, 3, 4, 5, 6, 7, 8, 9, 10, 11 or more peptide sequences capable of raising a humoral (antibody) and/or cellular immune response in vivo when administered to an immunocompetent subject. In an embodiment, the polyprotein comprises at least 1, preferably at least 2, preferably at least 3, preferably at least 4, preferably at least 5, preferably at least 6, preferably at least 7, preferably at least 8, or more preferably at least 9 immunogens. In an embodiment where the polyprotein comprises at least 2 immunogens, each immunogen may suitably be capable of raising a humoral (antibody) and/or cellular immune response to a different target antigen in vivo. For example, the polyprotein may comprise a first immunogen comprising a B cell epitope and/or a T cell epitope of subunit 1 of the SARS-CoV-2 spike protein and a second immunogen comprising a B cell epitope and/or a T cell epitope of subunit 2 of the SARS-CoV-2 spike protein. In other illustrative examples, the at least two immunogens may be capable of raising an immune response to the same target antigen. For instance, the polyprotein may comprise a first immunogen comprising a B cell epitope of subunit 1 of the SARS-CoV-2 spike protein and a second immunogen comprising a T cell epitope of subunit 1 of the SARS-CoV-2 spike protein. Thus, some embodiments, the polyprotein comprises 2 or more immunogens capable of raising a humoral and/or cellular immune response to the same target antigen in vivo. Advantageously, the immunogen will be capable of raising a humoral and/or cellular immune response to an extracellular portion of the native (e.g., wild-type) target antigen in vivo.

As noted elsewhere herein, the immunogen comprises a B cell epitope and/or a T cell epitope of a SARS-CoV surface protein. Suitable SARS-CoV surface proteins (including SARS-CoV-2 surface proteins) will be familiar to persons skilled in the art, illustrative examples of which are described elsewhere wherein. In an embodiment, the SARS-CoV surface protein is a SARS-CoV-2 surface protein. In an embodiment, the SARS-CoV surface protein is selected from the group consisting of:
(i) Subunit 1 of SARS-CoV-2 spike protein (S1) comprising an amino acid sequence of SEQ ID NO:3 or an amino acid having at least 85% sequence identity thereto; and
(ii) Subunit 2 of SARS-CoV-2 spike protein (S2) comprising an amino acid sequence of SEQ ID NO:4 or an amino acid having at least 85% sequence identity thereto.

In an embodiment, the immunogen comprises a B cell epitope and/or a T cell epitope of a receptor binding domain of SARS-CoV. In an embodiment, the receptor binding domain of SARS-CoV comprises an amino acid sequence of SEQ ID NO:5, or an amino acid having at least 85% sequence identity thereto.

Reference to “at least 85% sequence identity” includes 85%, 86%, 87%, 88%, 89%, 90%, 91%, 92%, 93%, 94%, 95%, 96%, 97%, 98%, 99% or 100% sequence identity to a reference sequence, for example, after optimal alignment or best fit analysis. In an embodiment, the amino acid sequence of the SARS-CoV surface protein has at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to the corresponding reference sequence, after optimal alignment or best fit analysis.

Where the polyprotein encoded by the nucleic acid construct comprises at least two immunogens, the at least two immunogens may have a branched or linear configuration, including as a fusion protein. As used herein, the term “fusion protein” typically refers to a polypeptide or polyprotein composed of two or more peptide or protein sequences linked to one another. In an embodiment, the fusion protein comprises two or more peptide or protein sequences linked to one another end-to-end. In an embodiment, the fusion protein comprises two or more peptide or protein sequences linked to one another in a linear configuration via a suitable linking moiety, also referred to herein as a linker. Suitable methods of linking peptide sequences will be familiar to persons skilled in the art, illustrative examples of which include peptide (amide) bonds and linkers. As used herein, the term “linker” refers to a short polypeptide sequence interposed between any two neighboring peptide sequences as herein described. In an embodiment, the linker is a polypeptide linker of 1 to 10 amino acids, preferably 1, 2, 3, 4 or 5 naturally or non-naturally occurring amino acids. In an embodiment, the linker is a carbohydrate linker. Suitable carbohydrate linkers will be known to persons skilled in the art. In another embodiment disclosed herein, the fusion protein comprises one or more peptidic or polypeptidic linker(s) together with one or more other non-peptidic or non-polypeptidic linker(s). Further, different types of linkers, peptidic or non-peptidic, may be incorporated in the same fusion peptide as deemed appropriate. In the event that a peptidic or polypeptidic linker is used to join two respective peptide sequences, the linker will be advantageously incorporated such that its N-terminal end is bound via a peptide bond to the C-terminal end of the one peptide sequence, and its C-terminal end via a peptide bond to the N-terminal end of the other peptide sequence. The individual peptide sequences within the fusion protein may also have one or more amino acids added to either or both ends, preferably to the C-terminal end. Thus, for example, linker or spacer amino acids may be added to the N- or C-terminus of the peptides or both, to link the peptides and to allow for convenient coupling of the peptides to each other and/or to a delivery system such as a carrier molecule serving as an anchor. An illustrative example of a suitable peptidic linker is LP (leucine-proline). The immunogen may suitably comprise a fusion protein comprising, consisting, or consisting essentially of any combination of two or more of the peptide sequences. In an embodiment, the fusion protein comprises, consists, or consists essentially of at least three of the peptide sequences. Also contemplated herein are fusion proteins comprising at least two of the peptide sequences disclosed herein, concatenated two or more times in tandem repeat.

Without being bound by theory or by a particular mode of application, it will be understood that incorporating two or more different immunogens into the VLP, as herein described, may suitably generate a more beneficial immune response by eliciting a higher antibody titre or enhanced immune cell activation as compared to a VLP comprising a single immunogen.

It is to be understood that the immunogen, as described herein, may comprise an amino acid sequence of any suitable length, as long as the immunogen retains the ability or capacity to induce an immune response in vivo to the target antigen.

As used herein, the term “B cell epitope” refers to a part of a molecule that is recognized by an antibody. Thus, a “B cell epitope” is to be understood as being a smaller subsequence of an antigen that is capable of being recognized (bound) by an antibody. It is to be understood that an antigen may contain multiple B cell epitopes, and therefore may be bound by multiple distinct antibodies. A single epitope may also be bound by multiple antibodies having different antigen-binding specificity and/or affinity. Multiple antibodies of different subclasses may also bind to the same epitope.

In an embodiment, the immunogen will comprise at least one peptide sequence or protein that, when administered to a subject, will induce an antibody response such that the antibody binds to a B cell epitope of the target antigen, preferably to a B cell epitope of a native target antigen; that is, to the target antigen as it exists in nature. Preferably, the at least one peptide or protein sequence of the immunogen will induce an antibody response such that the antibody binds to a B cell epitope of the extracellular domain of a native target antigen. Preferably, the B cell epitope to which an antibody is raised will be an epitope located within the extracellular domain of the native target antigen. In an embodiment, the at least one immunogen comprises an autologous B cell epitope of the target antigen; that is, a B cell epitope of the target antigen having an amino acid sequence derived from a target antigen of the same species as the subject to be treated.

Methods of determining the B cell epitope of a target antigen will be familiar to persons skilled in the art. In an illustrative example, a sub-sequence of an antigen may be identified with a high degree of accuracy as being, or comprising, a B cell epitope by using established computer programs that compare the subsequence in question with a database of known sequences and/or partial sequences known to be recognized by antibodies encoded by the human or mouse germline. Alternatively, a B cell epitope may be identified by computer-aided analysis using various combinations of correlates of antigenicity such as surface accessibility, chain flexibility, hydropathy/hydrophilicity profiles, predicted secondary structure, etc. Alternatively, an antigen may be identified as comprising a B cell epitope by immunising an animal with the antigen in question at least once, allowing a humoral immune response to mount and then testing the serum of the animal for antibodies that specifically bind to at least a part of the administered antigen using, for example, an enzyme linked immunosorbant assay (ELISA), a radioimmunoassay, a Western blot analysis or a dot-blot analysis.

As used herein, the term “T cell epitope” means an epitope presented on the surface of an antigen-presenting cell bound to an MHC molecule. T cell epitopes presented by MHC class I molecules are typically peptides between 8 and 11 amino acids in length, whereas MHC class II molecules are present as longer peptides, typically 13-17 amino acids in length.

In an embodiment, the immunogen will comprise at least one peptide sequence that, when administered to a subject, will induce a T cell response towards the target antigen, preferably to a T cell epitope of a native target antigen; that is, to the target antigen as it exists in nature. B cell and T cell epitopes of target antigens can be identified by persons skilled in the art using known methodologies, illustrative examples of which are described in Sanchez-Trincado et al. (Journal of Immunology Research, 2017; article ID 2680160).

As used herein, the terms “native” and “natural” refer to a sequence that is normally found in nature. Conversely, a “non-native” sequence, including a “non-native linker” is any amino acid sequence not belonging to native sequence of the target antigen.

The present disclosure also extends to the use of immunogens that are a “functional variant” of the native target sequence. It is to be understood that a “functional variant”, as used herein, means a peptide sequence that has a different amino acid sequence to a peptide to which it is compared (i.e., a comparator), which may include a natural (i.e., native) sequence or a synthetic variant thereof, yet retains the ability to induce a humoral and/or cellular immune response in vivo to the target antigen.

Suitable methods of determining whether a functional variant retains the ability to induce a humoral and/or cellular immune response in vivo against the native target antigen will be familiar to persons skilled in the art, illustrative examples of which are described elsewhere herein. A functional variant may include an amino acid sequence that differs from the native peptide sequence by one or more (e.g., 1, 2, 3, 4, 5, 6, 7, 8, 9 or more) amino acid substitutions, wherein said difference does not, or does not completely, abolish the capacity of the variant to induce a humoral and/or cellular immune response towards the target antigen. In some embodiments, the functional variant may comprise amino acid substitutions that enhance the capacity of the peptide sequence to induce a humoral and/or cellular immune response to the target antigen, as compared to the native peptide sequence. In an embodiment, the functional variant differs from the native peptide sequence by one or more conservative amino acid substitutions. As used herein, the term “conservative amino acid substitution” refers to changing amino acid identity at a given position to replace it with an amino acid of approximately equivalent size, charge and/or polarity. Examples of natural conservative substitutions of amino acids include the following 8 substitution groups (designated by the conventional one-letter code): (1) M, I, L, V; (2) F, Y, W; (3) K, R, (4) A, G; (5) S, T; (6) Q, N; (7) E, D; and (8) C, S.

In an embodiment, a functional variant includes amino acid substitutions and/or other modifications in order to increase the stability of the immunogen and/or to increase the solubility of the immunogen to enhance its ability to induce an antibody response in vivo. Suitable modifications will be familiar to persons skilled in the art, illustrative examples of which are described elsewhere herein.

In an embodiment, the immunogen comprises a promiscuous T helper (Th) cell epitope. Suitable promiscuous Th cell epitopes will be known to persons skilled in the art, illustrative examples of which include measles virus fusion protein (MVF; KLLSLIKGVIVHRLEGVE; SEQ ID NO:9), tetanus toxoid (TT; NSVDDALINSTIYSYFPSV; SEQ ID NO:10), TT1 (PGINGKAIHLVNNQSSE; SEQ ID NO:11); TT peptide P2 (QYIKANSKFIGITEL; SEQ ID NO:12); TT peptide P30 (FNNFTVSFWLRVPKVSASHLE; SEQ ID NO:13); MVF′ (LSEIKGVIVHRLEGV; SEQ ID NO:14); Hepatitis B virus (HBV; FFLLTRILTIPQSLN; SEQ ID NO:15); circumsporozoite protein (CSP; TCGVGVRVRSRVNAANKKPEl; SEQ ID NO:16), and those described in WO2000/046390, the entire contents of which is incorporated herein by reference. In an embodiment, the promiscuous Th epitope is from about 8 to about 36, preferably from about 8 to about 24, more preferably from about 8 to 22, most preferably from about 8 to about 22 amino acids in length. In an embodiment, the promiscuous Th cell epitope is linked to a peptide sequences or fusion protein, as herein described, to form a chimeric peptide. Suitable linkers will be familiar to persons skilled in the art, illustrative examples of which are described elsewhere herein.

As noted elsewhere herein, the immunogen will comprise at least one peptide sequence that is capable of inducing a humoral and/or cellular immune response towards the native target antigen. This may be achieved by using a peptide sequence comprising an amino acid sequence of a B cell epitope and/or a T cell epitope of the target antigen (or a functional variant thereof), and/or a mimotope thereof. As used herein, the term “mimotope” refers to a molecule that has a conformation that has a topology equivalent to the B cell epitope or the T cell epitope of which it is a mimic such that it is capable of raising a humoral and/or cellular immune response that targets the same epitope of the native target antigen. In other words, when administered to a host, the VLP comprising the mimotope will elicit an immune response (humoral and/or cellular) in a host towards the target antigen of which it is a mimic

Peptide sequences disclosed herein can be synthetically produced by chemical synthesis methods which are well known in the art, either as an isolated peptide sequence or as a part of another peptide or polypeptide. Alternatively, peptide or protein sequences can be produced in a microorganism which produces the (recombinant) peptide or protein sequence or sequences, which can then be isolated and, if desired, further purified. The peptide or protein sequences can be produced in microorganisms such as bacteria, yeast or fungi, in eukaryote cells such as a mammalian or an insect cell, or in a recombinant virus vector such as adenovirus, poxvirus, herpesvirus, Simliki forest virus, baculovirus, bacteriophage, sindbis virus or sendai virus. Suitable bacteria for producing the peptide or protein sequences will be familiar to persons skilled in the art, illustrative examples of which include E. coli, B. subtilis or any other bacterium that is capable of expressing the peptide sequences. Illustrative examples of suitable yeast types for expressing the peptide or protein sequences include Saccharomyces cerevisiae, Schizosaccharomyces pombe, Candida, Pichia pastoris or any other yeast capable of expressing peptides. Corresponding methods are well known in the art. Also methods for isolating and purifying recombinantly produced peptide sequences are well known in the art and include, for example, gel filtration, affinity chromatography and ion exchange chromatography.

In some embodiments, the immunogen may comprise a peptide sequence that is heterologous (i.e., non-native) to the VLP; that is, it is heterologous to the virus from which the components (including structural components) of the VLP are derived. Alternatively, or in addition, the immunogen may advantageously be a component of any one or more of the viral structural proteins encoded by the nucleic acid construct described herein.

It is to be understood that, in some embodiments, the immunogen is heterologous (i.e., non-native) to the VLP; in other words, the two or more viral structural proteins comprise sequences derived from the structural proteins of a non-SARS-CoV virus. Suitable the structural proteins of a non-SARS-CoV virus that may be used to generate a VLP for the purposes of carrying the immunogen, as described herein, will be familiar to persons skilled in the art, illustrative examples of which include structural proteins of viruses of the family Flaviviridae, Orthomyxoviridae and Togaviridae. However, without being limited by theory or to a particular mode of action, it may be generally more convenient to generate a VLP by using structural proteins of the SARS-CoV, including of a SARS-CoV-2, in particular where the immunogen is a component of one of the two or more viral structural proteins of the polyprotein described herein.

### Viral Structural Proteins

The polypeptide may comprises, consists or consists essentially of at least two (e.g., 2, 3, 4, 5 and so on) viral structural proteins. In some embodiments, the polypeptide comprises, consists or consists essentially of at least three, preferably at least four, more preferably at least five viral structural proteins. In an embodiment, the polypeptide comprises, consists or consists essentially of three viral structural proteins. In another embodiment, the polypeptide comprises, consists or consists essentially of four viral structural proteins. In another embodiment, the polypeptide comprises, consists or consists essentially of five viral structural proteins. In another embodiment, the polypeptide comprises, consists or consists essentially of six viral structural proteins. It is to be understood that the polypeptide may comprise any number of two or more viral structural proteins, including any combination of viral capsid and/or envelope proteins, as long as the two or more viral structural proteins, once liberated following host cell peptidase-dependent cleavage, are capable of self-assembly to form a VLP carrying the immunogen. As noted elsewhere herein, each of the two or more viral structural proteins of the polyprotein, as described herein, once liberated following host cell peptidase-dependent cleavage, are capable of self-assembling into a VLP carrying the immunogen. In this context, viral structural proteins derived from any suitable virus can be used, as long as the viral structural proteins are capable of self-assembling into a VLP following host cell peptidase-dependent cleavage and liberation of the two or more viral structural proteins from the polyprotein. Suitable viral structural proteins will be familiar to persons skilled in the art, illustrative examples of which include structural proteins derived from viruses of the family Flaviviridae, Coronaviridae, Orthomyxoviridae and Togaviridae. Thus, in an embodiment, the viral structural proteins are derived from a virus of the family selected from the group consisting of Flaviviridae, Coronaviridae, Orthomyxoviridae and Togaviridae. In an embodiment, the viral structural proteins are derived from a virus of the family Coronaviridae (see Payne, S: Chapter 17—Family Coronaviridae; Viruses: From Understanding to Investigation, 2017, Pages 149-158; and Family—Coronaviridae: Virus Taxonomy, Ninth Report of the International Committee on Taxonomy of Viruses, 2012, Pages 806-828). The Coronaviridae family is typically divided into Coronavirinae and Torovirinae sub-families, which are further divided into six genera: Alphacoronavirus, Betacoronavirus, Gammacoronavirus, Deltacoronavirus, Torovirus, and Bafinivirus. While viruses in the genera Alphacoronaviruses and Betacoronaviruses infect mostly mammals, the Gamma-coronavirus infect avian species and members of the Deltacoronavirus genus have been found in both mammalian and avian hosts (see, e.g., Phan et al., Virus Evol. 2018; 4(2): vey035). Coronaviruses (CoV) cause a range of respiratory, enteric, and neurological diseases in human and animals. In human CoV infections, the severe acute respiratory syndrome coronavirus (SARS-CoV) and the severe acute respiratory syndrome coronavirus 2 (SARS-CoV-2) can cause severe respiratory tract disease with high mortality rates.

Members of the Coronaviridae family share the same unique strategy for mRNA synthesis whereby the polymerase complex jumps or moves from one region of the template to a more distant region. The need for the polymerase complex to dissociate from the template may explain the high rate of RNA recombination that occurs during genome replication. In the present context, suitable viruses of the family Coronaviridae will be familiar to persons skilled in the art, illustrative examples of which include Alphaletovirus (see, e.g., Bukhari et al.; Virology. 2018; 524:160-171) and Coronavirus (see, e.g., Fehr and Perlman; Coronaviruses. 2015; 1282: 1-23). Thus, in an embodiment disclosed herein, the virus is selected from the group consisting of Alphaletovirus and Coronavirus. In an embodiment disclosed herein, the Coronavirus is selected from the group consisting of Alphacoronavirus, Betacoronavirus, Deltacoronavirus and Gammacoronavirus. In an embodiment, the Coronavirus is Betacoronavirus. Suitable Betacoronaviruses will be familiar to persons skilled in the art, an illustrative example of which includes a Sarbecovirus. Thus, in an embodiment, the Betacoronavirus is a Sarbecovirus.

Suitable Sarbecoviruses will be familiar to persons skilled in the art, illustrative examples of which include Severe acute respiratory syndrome-related coronavirus, Severe acute respiratory syndrome coronavirus (SARS-CoV; see, e.g., Vijayanand et al., Clin Med (Lond). 2004; 4(2):152-60) and Severe acute respiratory syndrome coronavirus 2 (SARS-CoV-2; see, e.g., Khailany et al. Gene Rep. 2020; 19: 100682). In an embodiment disclosed herein, the Sarbecovirus is selected from the group consisting of Severe acute respiratory syndrome-related coronavirus, SARS-CoV and SARS-CoV-2. In an embodiment, the Sarbecovirus is SARS-CoV-2. As reported by Khailany et al. (ibid), the recent outbreak of coronavirus disease (COVID-19) that was first reported from Wuhan, China, in December 2019. SARS-CoV-2 is transmitted from person to person via droplet transmission and is therefore easily spread in overcrowded areas. Most patients experience only mild to moderate symptoms, such as high body temperature in conjunction with some respiratory symptoms such as cough, sore throat, and headache. Some people may have severe symptoms like pneumonia and acute respiratory distress syndrome. Notably, individuals with underlying complications such as heart disease, chronic lung disease, or diabetes potentially display more severe symptoms. To date, no specific antiviral treatment is proven effective, hence, infected people initially rely on symptomatic treatments that showed encouraging profile for blocking the new coronavirus in early clinical trials.

The genome SARS-CoV-2 varies from 29.8 kb to 29.9 kb and has a structure that is typical of other known coronaviruses, insofar as at the 5′ more than two-thirds of the genome comprises orf1ab encoding orf1abpolyproteins, while at the 3′ one third consists of genes encoding structural proteins including surface (S), envelope (E), membrane (M), and nucleocapsid N proteins (see GenBank Accession No. NC_045512, the entire contents of which is incorporated herein by reference). Additionally, the SARS-CoV-2 contains 6 accessory proteins, encoded by ORF3a, ORF6, ORF7a, ORF7b, and ORF8.

In some contexts, it may be desirable to include two or more copies of the same viral structural protein, as described herein. Without being bound by theory or by a particular mode of application, this approach may provide additional immunogen against which an immune response can be generated in a subject in which the vaccine construct or VLP is to be administered.

In an embodiment disclosed herein, the SARS-CoV is SARS-CoV-2. As reported by Khailany et al. (ibid), the first outbreak of coronavirus disease (COVID-19) was reported from Wuhan, China, in December 2019. SARS-CoV-2 is transmitted from person to person via droplet transmission and is therefore easily spread in overcrowded areas. Most patients experience only mild to moderate symptoms, such as high body temperature in conjunction with some respiratory symptoms such as cough, sore throat, and headache. Some people may have severe symptoms like pneumonia and acute respiratory distress syndrome. Notably, individuals with underlying complications such as heart disease, chronic lung disease, or diabetes potentially display more severe symptoms. To date, no specific antiviral treatment is proven effective, hence, infected people have largely relied on symptomatic treatments that showed encouraging profile for blocking the new coronavirus.

The genome SARS-CoV-2 varies from 29.8 kb to 29.9 kb and has a structure that is typical of other known coronaviruses, insofar as at the 5′ more than two-thirds of the genome comprises orf1ab encoding orf1abpolyproteins, while at the 3′ one third consists of genes encoding structural proteins including surface or spike protein (S), envelope protein (E), membrane protein (M), and nucleocapsid N protein (see GenBank Accession No. NC_045512, the entire contents of which is incorporated herein by reference). Additionally, the SARS-CoV-2 contains 6 accessory proteins, encoded by ORF3a, ORF6, ORF7a, ORF7b, and ORF8.

The total length of SARS-CoV-2 S is around 1273 amino acids (SEQ ID NO:1; see below) and consists of a signal peptide (amino acid residues 1-13 of SEQ ID NO:1) located at the N-terminus, the S1 subunit (amino acid residues 14-685 of SEQ ID NO:1), and the S2 subunit (amino acid residues 686-1273 of SEQ ID NO:1). Subunits 1 and 2 of the S protein are responsible for receptor binding and membrane fusion, respectively. In the S1 subunit, there is an N-terminal domain (amino acid residues 14-305 of SEQ ID NO:1) and a receptor-binding domain (RBD, amino acid residues 319-541 of SEQ ID NO:1); the fusion peptide (FP) (amino acid residues 788-806 of SEQ ID NO:1), heptapeptide repeat sequence 1 (HR1) (amino acid residues 912-984 of SEQ ID NO:1), HR2 (amino acid residues 1163-1213 of SEQ ID NO:1), TM domain (amino acid residues 1213-1237 of SEQ ID NO:1), and cytoplasm domain (amino acid residues 1237-1273 of SEQ ID NO:1) make up the S2 subunit.

### SARS-CoV-2 Surface or Spike Protein (SEQ ID NO:1; NCBI Reference Sequence: YP_009724390)

MFVFLVLLPLVSSQCVNLTTRTQLPPAYTNSFTRGVYYPDKVFRS
SVLHSTQDLFLPFFSNVTWFHAIHVSGTNGTKRFDNPVLPFNDGV
YFASTEKSNIIRGWIFGTTLDSKTQSLLIVNNATNVVIKVCEFQF
CNDPFLGVYYHKNNKSWMESEFRVYSSANNCTFEYVSQPFLMDLE
GKQGNFKNLREFVFKNIDGYFKIYSKHTPINLVRDLPQGFSALEP
LVDLPIGINITRFQTLLALHRSYLTPGDSSSGWTAGAAAYYVGYL
QPRTFLLKYNENGTITDAVDCALDPLSETKCTLKSFTVEKGIYQT
SNFRVQPTESIVRFPNITNLCPFGEVFNATRFASVYAWNRKRISN
CVADYSVLYNSASFSTFKCYGVSPTKLNDLCFTNVYADSFVIRGD
EVRQIAPGQTGKIADYNYKLPDDFTGCVIAWNSNNLDSKVGGNYN
YLYRLFRKSNLKPFERDISTEIYQAGSTPCNGVEGFNCYFPLQSY
GFQPTNGVGYQPYRVVVLSFELLHAPATVCGPKKSTNLVKNKCVN
FNFNGLTGTGVLTESNKKFLPFQQFGRDIADTTDAVRDPQTLEIL
DITPCSFGGVSVITPGTNTSNQVAVLYQDVNCTEVPVAIHADQLT
PTWRVYSTGSNVFQTRAGCLIGAEHVNNSYECDIPIGAGICASYQ
TQTNSPRRARSVASQSIIAYTMSLGAENSVAYSNNSIAIPTNFTI
SVTTEILPVSMTKTSVDCTMYICGDSTECSNLLLQYGSFCTQLNR
ALTGIAVEQDKNTQEVFAQVKQIYKTPPIKDFGGFNFSQILPDPS
KPSKRSFIEDLLFNKVTLADAGFIKQYGDCLGDIAARDLICAQKF
NGLTVLPPLLTDEMIAQYTSALLAGTITSGWTFGAGAALQIPFAM
QMAYRFNGIGVTQNVLYENQKLIANQFNSAIGKIQDSLSSTASAL
GKLQDVVNQNAQALNTLVKQLSSNFGAISSVLNDILSRLDKVEAE
VQIDRLITGRLQSLQTYVTQQLIRAAEIRASANLAATKMSECVLG
QSKRVDFCGKGYHLMSFPQSAPHGVVFLHVTYVPAQEKNFTTAPA
ICHDGKAHFPREGVFVSNGTHWFVTQRNFYEPQIITTDNTFVSGN
CDVVIGIVNNTVYDPLQPELDSFKEELDKYFKNHTSPDVDLGDIS
GINASVVNIQKEIDRLNEVAKNLNESLIDLQELGKYEQYIKWPWY
IWLGFIAGLIAIVMVTIMLCCMTSCCSCLKGCCSCGSCCKFDEDD
SEPVLKGVKLHYT

### SARS-CoV-2 S Protein Signal Peptide (SEQ ID NO:2)

MFVFLVLLPLVS

### SARS-CoV-2 S Protein S1 Subunit (SEQ ID NO:3; Underlined Sequence is the Receptor Binding Domain, Also Shown as SEQ ID NO:5 Below)

SQCVNLTTRTQLPPAYTNSFTRGVYYPDKVFRSSVLHSTQDLFLP
FFSNVTWFHAIHVSGTNGTKRFDNPVLPFNDGVYFASTEKSNIIR
GWIFGTTLDSKTQSLLIVNNATNVVIKVCEFQFCNDPFLGVYYHK
NNKSWMESEFRVYSSANNCTFEYVSQPFLMDLEGKQGNFKNLREF
VFKNIDGYFKIYSKHTPINLVRDLPQGFSALEPLVDLPIGINITR
FQTLLALHRSYLTPGDSSSGWTAGAAAYYVGYLQPRTFLLKYNEN
GTITDAVDCALDPLSETKCTLKSFTVEKGIYQTSNFRVQPTESIV
RFPNITNLCPFGEVFNATRFASVYAWNRKRISNCVADYSVLYNSA
SFSTFKCYGVSPTKLNDLCFTNVYADSFVIRGDEVRQIAPGQTGK
IADYNYKLPDDFTGCVIAWNSNNLDSKVGGNYNYLYRLFRKSNLK
PFERDISTEIYQAGSTPCNGVEGFNCYFPLQSYGFQPTNGVGYQP
YRVVVLSFELLHAPATVCGPKKSTNLVKNKCVNFNFNGLTGTGVL
TESNKKFLPFQQFGRDIADTTDAVRDPQTLEILDITPCSFGGVSV
ITPGTNTSNQVAVLYQDVNCTEVPVAIHADQLTPTWRVYSTGSNV
FQTRAGCLIGAEHVNNSYECDIPIGAGICASYQTQTNSPRRAR

### SARS-CoV-2 S Protein S2 Subunit (SEQ ID NO:4)

SVASQSIIAYTMSLGAENSVAYSNNSIAIPTNFTISVTTEILPVS
MTKTSVDCTMYICGDSTECSNLLLQYGSFCTQLNRALTGIAVEQD
KNTQEVFAQVKQIYKTPPIKDFGGFNFSQILPDPSKPSKRSFIED
LLFNKVTLADAGFIKQYGDCLGDIAARDLICAQKFNGLTVLPPLL
TDEMIAQYTSALLAGTITSGWTFGAGAALQIPFAMQMAYRENGIG
VTQNVLYENQKLIANQFNSAIGKIQDSLSSTASALGKLQDVVNQN
AQALNTLVKQLSSNFGAISSVLNDILSRLDKVEAEVQIDRLITGR
LQSLQTYVTQQLIRAAEIRASANLAATKMSECVLGQSKRVDFCGK
GYHLMSFPQSAPHGVVFLHVTYVPAQEKNFTTAPAICHDGKAHFP
REGVFVSNGTHWFVTQRNFYEPQIITTDNTFVSGNCDVVIGIVNN
TVYDPLQPELDSFKEELDKYFKNHTSPDVDLGDISGINASVVNIQ
KEIDRLNEVAKNLNESLIDLQELGKYEQYIKWPWYIWLGFIAGLI
AIVMVTIMLCCMTSCCSCLKGCCSCGSCCKFDEDDSEPVLKGVKL
HYT

### SARS-CoV-2 S Protein Receptor Binding Domain (RBD) (SEQ ID NO:5)

RVQPTESIVRFPNITNLCPFGEVFNATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYLY
RLFRKSNLKPFERDISTEIYQAGSTPCNGVEGENCYFPLQSYGFQ
PTNGVGYQPYRVVVLSFELLHAPATVCGPKKSTNLVKNKCVNF

### SARS-CoV-2 Membrane Protein (SEQ ID NO:6)

MADSNGTITVEELKKLLEQWNLVIGFLFLTWICLLQFAYANRNRF
LYIIKLIFLWLLWPVTLACFVLAAVYRINWITGGIAIAMACLVGL
MWLSYFIASFRLFARTRSMWSFNPETNILLNVPLHGTILTRPLLE
SELVIGAVILRGHLRIAGHHLGRCDIKDLPKEITVATSRTLSYYK
LGASQRVAGDSGFAAYSRYRIGNYKLNTDHSSSSDNIALLVQ

### SARS-CoV-2 Envelope Protein (SEQ ID NO:7)

MYSFVSEETGTLIVNSVLLFLAFVVFLLVTLAILTALRLCAYCCN
IVNVSLVKPSFYVYSRVKNLNSSRVPDLLV

### SARS-CoV-2 Nucleoprotein (SEQ ID NO:8)

MSDNGPQNQRNAPRITFGGPSDSTGSNQNGERSGARSKQRRPQGL
PNNTASWFTALTQHGKEDLKFPRGQGVPINTNSSPDDQIGYYRRA
TRRIRGGDGKMKDLSPRWYFYYLGTGPEAGLPYGANKDGIIWVAT
EGALNTPKDHIGTRNPANNAAIVLQLPQGTTLPKGFYAEGSRGGS
QASSRSSSRSRNSSRNSTPGSSRGTSPARMAGNGGDAALALLLLD
RLNQLESKMSGKGQQQQGQTVTKKSAAEASKKPRQKRTATKAYNV
TQAFGRRGPEQTQGNFGDQELIRQGTDYKHWPQIAQFAPSASAFF
GMSRIGMEVTPSGTWLTYTGAIKLDDKDPNFKDQVILLNKHIDAY
KTFPPTEPKKDKKKKADETQALPQRQKKQQTVTLLPAADLDDFSK
QLQQSMSSADSTQA

The receptor binding domain (RBD) of SARS-CoV-2 is located in the S1 subunit and binds to the cell receptor angiotensin converting enzyme 2 (ACE2) and is a critical target for neutralizing antibodies. The SARS-CoV-2 RBD and the SARS-CoV RBD share around 73%-76% sequence identity. Nine ACE2-contacting residues in CoV RBD are fully conserved, and four are partially conserved. Analysis of the RBM (receptor-binding motif, a portion of RBD making direct contacts with ACE2) of SARS-CoV and SARS-CoV-2 reveals that most residues essential for ACE2 binding in the SARS-CoV S protein are conserved in the SARS-CoV-2 S protein. The S2 subunit, comprising an FP, HR1, HR2, TM domain, and cytoplasmic domain fusion (CT), is responsible for viral fusion and entry. FP is a short segment of 15-20 conserved amino acids of the viral family, composed mainly of hydrophobic residues, such as glycine (G) or alanine (A), which anchor to the target membrane when the S protein adopts the prehairpin conformation. The HR1 and HR2 domains are composed of a repetitive heptapeptide: HPPHCPC, where H is a hydrophobic or traditionally bulky residue, P is a polar or hydrophilic residue, and C is another charged residue. HR1 and HR2 form the six-helical bundle (6-HB), which is essential for the viral fusion and entry function of the S2 subunit. HR1 is located at the C-terminus of a hydrophobic FP, and HR2 is located at the N-terminus of the TM domain. The Downstream™ domain anchors the S protein to the viral membrane, and the S2 subunit ends in a CT tail. When the RBD binds to ACE2, the S2 changes conformation by inserting FP into the target cell membrane, exposing the prehairpin coiled-coil of the HR1 domain and triggering interaction between the HR2 domain and HR1 trimer to form 6-HB, thus bringing the viral envelope and cell membrane into proximity for viral fusion and entry. HR1 forms a homotrimeric assembly in which three highly conserved hydrophobic grooves on the surface that bind to HR2 are exposed. The HR2 domain forms both a rigid helix and a flexible loop to interact with the HR1 domain. The S protein on the surface of SARS-CoV-2 is a key factor involved in infection. It is a trimeric class I TM glycoprotein responsible for viral entry. Similar to other coronaviruses, the S protein of SARS-CoV-2 mediates receptor recognition, cell attachment, and fusion during viral infection. The trimer of the S protein located on the surface of the viral envelope is the basic unit by which the S protein binds to the receptor. The S1 domain contains the RBD, which is mainly responsible for binding of the virus to the receptor, while the S2 domain mainly contains the HR domain, including HR1 and HR2, which is closely related to virus fusion (Huang et al.; 2020; Acta Pharmacologica Sinica; 41:1141-1149).

The polyprotein, described herein, will suitably comprise a number and type of viral structural proteins that are sufficient to enable the structural proteins, once liberated from the polyprotein following host cell peptidase-dependent cleavage, to self-assemble into a VLP. In an embodiment, the polyprotein comprises at least three or more preferably at least four viral structural proteins of the SARS-CoV, including of a SARS-CoV-2. In an embodiment, the polyprotein comprises three viral structural proteins. In an embodiment, the polyprotein comprises four viral structural proteins.

In the context of the present disclosure, suitable structural proteins of the SARS-CoV will be familiar to persons skilled in the art, illustrative examples of which are described in Song et al. (2020, Genomics, Proteomics & Bioinformatics, “The Global Landscape of SARS-CoV-2 Genomes, Variants, and Haplotypes in 2019nCoVR.”). Nucleic acid and amino acid sequences of SARS-CoV strains and variants thereof are also accessible from the NIH genetic sequence database, GenBank. It is to be understood that the present disclosure is not limited to known strain of SARS-CoV, and is therefore applicable to emerging strain of SARS-CoV.

In an embodiment, the two or more viral structural proteins are selected from the group consisting of:
(i) Subunit 1 of SARS-CoV-2 spike protein (S1) comprising an amino acid sequence of SEQ ID NO:3 or an amino acid having at least 85% sequence identity thereto;
(ii) Subunit 2 of SARS-CoV-2 spike protein (S2) comprising an amino acid sequence of SEQ ID NO:4 or an amino acid having at least 85% sequence identity thereto;
(iii) SARS-CoV-2 membrane protein comprising an amino acid sequence of SEQ ID NO:6 or an amino acid having at least 85% sequence identity thereto;
(iv) SARS-CoV-2 envelope protein comprising an amino acid sequence of SEQ ID NO:7 or an amino acid having at least 85% sequence identity thereto; and
(v) SARS-CoV-2 nucleoprotein comprising an amino acid sequence of SEQ ID NO:8 or an amino acid having at least 85% sequence identity thereto.

In an embodiment, the polyprotein comprises:
(i) Subunit 1 of SARS-CoV-2 spike protein (S1) comprising an amino acid sequence of SEQ ID NO:3 or an amino acid having at least 85% sequence identity thereto;
(ii) Subunit 2 of SARS-CoV-2 spike protein (S2) comprising an amino acid sequence of SEQ ID NO:4 or an amino acid having at least 85% sequence identity thereto;
(iii) SARS-CoV-2 membrane protein comprising an amino acid sequence of SEQ ID NO:6 or an amino acid having at least 85% sequence identity thereto;
(iv) SARS-CoV-2 envelope protein comprising an amino acid sequence of SEQ ID NO:7 or an amino acid having at least 85% sequence identity thereto; and
(v) SARS-CoV-2 nucleoprotein comprising an amino acid sequence of SEQ ID NO:8 or an amino acid having at least 85% sequence identity thereto.

In an embodiment, the polyprotein comprises a B cell epitope of the native receptor binding domain of the SARS-CoV-2, or a mimotope thereof. In an embodiment, the S1 comprises a native receptor binding domain of SARS-CoV-2.

In some contexts, it may be desirable to include two or more copies of any of the two or more viral structural proteins, as described herein. Without being bound by theory or by a particular mode of application, this approach may provide additional immunogen against which an immune response can be generated in a subject in which the nucleic acid construct or VLP is to be administered. In an embodiment, the polyprotein comprises at least two SARS-CoV-2 S1 proteins.

As noted elsewhere herein, the present disclosure extends to the use of variants of the two or more viral structural proteins of the polypeptide described herein. The term “variant”, in this context, typically refers to a viral structural protein comprising an amino acid sequence having at least 80% sequence identity to the amino acid sequence of the reference (e.g., native) protein. Reference to “at least 80% sequence identity” includes 80%, 85%, 86%, 87%, 88%, 89%, 90%, 91%, 92%, 93%, 94%, 95%, 96%, 97%, 98%, 99% or 100% sequence identity to a reference sequence, for example, after optimal alignment or best fit analysis. In an embodiment, the amino acid sequence of the structural protein has at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to the corresponding reference sequence, after optimal alignment or best fit analysis.

Reference to “at least 80% sequence identity” therefore suitably includes 80%, 85%, 86%, 87%, 88%, 89%, 90%, 91%, 92%, 93%, 94%, 95%, 96%, 97%, 98%, 99% or 100% sequence identity any one of SEQ ID NOs:1 and 3-8, for example, after optimal alignment or best fit analysis. In an embodiment, the variant has at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to any one of SEQ ID NOs:1 and 3-8, for example, after optimal alignment or best fit analysis.

Where a variant of a viral structural protein is employed in the polyprotein disclosed herein, the variant will suitably be a “functional variant”. The term “functional variant”, as used herein, typically means a peptide sequence that has a different amino acid sequence to the reference sequence to which it is compared, including a natural (i.e., native) sequence or a synthetic variant thereof, yet retains at least some of the function ascribed to the reference molecule, such as its ability to self-assemble with the other liberated viral structural protein(s) of the polyprotein to form a VLP and/or raise an immune response against the native protein, as described herein. Suitable methods of determining whether a variant retains said function will be familiar to persons skilled in the art, illustrative examples of which are described elsewhere herein. A functional variant may include an amino acid sequence that differs from the reference sequence (e.g., a native sequence) by one or more (e.g., at least 1, at least 2, at least 3, at least 4, at least 5, etc) amino acid substitutions, deletions or insertions, wherein said difference does not, or does not completely, abolish the ability of the variant to (i) self-assemble with the other liberated viral structural protein(s) of the polyprotein to form a VLP and/or (ii) raise an immune response against the native protein, as described herein. In some embodiments, the functional variant may comprise amino acid substitutions that enhance the ability of the variant to (i) self-assemble with the other liberated viral structural protein(s) of the polyprotein to form a VLP and/or (ii) raise an immune response against the native protein, as described herein self.

### Signal Peptidase Sequences

The terms “signal peptidase sequence” and “signalase sequence” are used interchangeably herein to mean an amino acid sequence that is specifically recognized and cleaved by peptidases. In the context of the present disclosure, the signal peptidase sequences are recognized and cleaved by host cell peptidases, also referred to herein as “signal peptide peptidases”. This advantageously allows the signal peptidase sequences to be cleaved by peptidases produced by the host cell. The peptidases may suitably be endogenous (i.e., native) to the host cell. Alternatively, or in addition, the host cell may be modified to produce a recombinant or heterologous peptidase. Conveniently, however, the host cell peptidase will be endogenous to the host cell, thereby avoiding the need to further modify the host cell to produce a recombinant peptidase. Thus, in an embodiment, the peptidase is native to the host cell. In other embodiments, the peptidase is a recombinant peptidase; that is, the peptidase is heterologous to the host cell.

The term “host cell peptidase-dependent cleavage” is to be understood to mean that the signal peptidase sequence(s) within the polyprotein, as described herein, is susceptible to cleavage by one or more peptidases (signal peptide peptidases) expressed by the host cell, whether the one or more peptidases are native to the host cell or recombinantly expressed by the host cell, as described elsewhere herein. In an embodiment, the signal peptidase sequence is susceptible to cleavage by one or more peptidases that are native to the host cell. Suitably, the signal peptidase sequence is not susceptible to virus peptidase-dependent or protease-dependent cleavage.

In an embodiment, the signal peptidase sequence is susceptible to host cell-dependent peptidases cleavage within the endoplasmic reticulum (ER) of the host cell. Suitable ER peptidases will be familiar to persons skilled in the art, illustrative examples of which are described in Oehler et al. (2012; J. Virol. 86(15):7818-7828), the entire contents of which are incorporated herein by reference.

It will be understood that the signal peptidase sequence will depend on the host cell peptidase to be employed to liberate the two or more viral structural proteins of the expressed polyprotein. Suitable signal peptidase sequences will be familiar to persons skilled in the art, illustrative examples of which include HCV core-E1 signal peptidase sequences, as described in Oehler et al. (2012; J. Virol. 86(15):7818-7828). In an embodiment, the signal peptidase sequence is a signal peptidase sequence utilized by hepatitis C virus, or a cleavable variant thereof. The term “cleavable variant” is to be understood to mean a variant (also referred to herein as a “functional variant” that has a different amino acid sequence to the peptide to which it is compared (i.e., a comparator or reference sequence), which may include a natural (i.e., native) sequence or a synthetic variant thereof, yet retains the ability to be cleaved by a host cell peptidase. In an embodiment, the signal peptidase sequence is selected from the group consisting of SEQ ID NOs:17-24 and amino acid sequences having at least 80% sequence identity to any of the foregoing.

### Core-E1 Signal Peptidase Sequences of Different HCV Genotypes:

GT 1a
(SEQ ID NO: 17)
GCSFSIFLLALLSCLTVPASA
GT 1b
(SEQ ID NO: 18)
GCSFSIFLLALLSCLTIPASA
GT 2a
(SEQ ID NO: 19)
GFPFSIFLLALLSCITVPVSA
GT 3a
(SEQ ID NO: 20)
GCSFSVFLLALFSCLIHPAAS
GT 4a
(SEQ ID NO: 21)
GCSFSIFLLALLSCLTVPASA
GT 5a
(SEQ ID NO: 22)
GCSFSIFILALLSCLTVPTSA
GT 6a
(SEQ ID NO: 23)
GCSFSIFLLALLSCLTTPASA
GT 7a
(SEQ ID NO: 24)
GCSFSIFLLALLSCLTVPASA

In an embodiment, the signal peptidase sequence comprises, consists or consists essentially of the amino acid sequence of SEQ ID NO:17 or an amino acid sequence having at least 80% sequence identity thereto.

Reference to “at least 80% sequence identity” includes 80%, 81%, 82%, 83%, 84%, 85%, 86%, 87%, 88%, 89%, 90%, 91%, 92%, 93%, 94%, 95%, 96%, 97%, 98%, 99% or 100% sequence identity any one of SEQ ID NOs:17-24, for example, after optimal alignment or best fit analysis. In an embodiment, the signal peptidase sequence has at least 80%, preferably at least 81%, preferably at least 82%, preferably at least 83%, preferably at least 84%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to any one of SEQ ID NOs:17-24, for example, after optimal alignment or best fit analysis.

Where the signal peptidase sequence is a variant having an amino acid sequence having at least 80% sequence identity to any one of SEQ ID NO:17-24, the variant will suitably be a “functional variant” (e.g., of the native sequence). It is to be understood that a “functional variant”, as used herein, means a peptide sequence that has a different amino acid sequence to the peptide to which it is compared (i.e., a comparator or reference sequence), which may include a natural (i.e., native) sequence or a synthetic variant thereof, yet retains the ability to be cleaved by host cell peptidase, as described herein. Thus, in this context, the variant is also referred to herein as a cleavable variant.

Suitable methods of determining whether a variant retains said function will be familiar to persons skilled in the art, illustrative examples of which are described elsewhere herein. A functional variant may include an amino acid sequence that differs from the reference sequence (e.g., any one of SEQ ID NOs:17-24) by at least one (e.g., 1, 2, 3, 4 or 5) amino acid substitutions, deletions or insertions, wherein said difference does not, or does not completely, abolish the ability of the variant to undergo host cell peptidase-depended cleavage. In some embodiments, the functional variant may comprise amino acid substitutions that enhance the ability of the variant to undergo host cell peptidase-depended cleavage, as compared to the native signal peptidase sequence. In an embodiment, the functional variant differs from the native signal peptidase sequence by one or more conservative amino acid substitutions. As used herein, the term “conservative amino acid substitution” refers to changing amino acid identity at a given position to replace it with an amino acid of approximately equivalent size, charge and/or polarity. Examples of natural conservative substitutions of amino acids include the following 8 substitution groups (designated by the conventional one-letter code): (1) M, I, L, V; (2) F, Y, W; (3) K, R, (4) A, G; (5) S, T; (6) Q, N; (7) E, D; and (8) C, S.

As used herein, the terms “identity”, “similarity”, “sequence identity”, “sequence similarity”, “homology”, “sequence homology” and the like mean that at any particular amino acid residue position in an aligned sequence, the amino acid residue is identical between the aligned sequences. The term “similarity” or “sequence similarity” as used herein, indicates that, at any particular position in the aligned sequences, the amino acid residue is of a similar type between the sequences. For example, leucine may be substituted for an isoleucine or valine residue. This may be referred to as conservative substitution. In an embodiment, the amino acid sequences may be modified by way of conservative substitution of any of the amino acid residues contained therein, such that the modification has no effect on the function of the modified polypeptide or polyprotein when compared to the unmodified polypeptide or polyprotein.

In some embodiments, sequence identity with respect to a peptide sequence relates to the percentage of amino acid residues in the candidate sequence which are identical with the residues of the corresponding peptide sequence after aligning the sequences and introducing gaps, if necessary, to achieve the maximum percentage homology, and not considering any conservative substitutions as part of the sequence identity. Neither N- or C-terminal extensions, nor insertions shall be construed as reducing sequence identity or homology. Methods and computer programs for performing an alignment of two or more amino acid sequences and determining their sequence identity or homology are well known to persons skilled in the art. For example, the percentage of identity or similarity of two amino acid sequences can be readily calculated using algorithms, for example, BLAST, FASTA, or the Smith-Waterman algorithm.

Techniques for determining an amino acid sequence “similarity” are well known to persons skilled in the art. In general, “similarity” means an exact amino acid to amino acid comparison of two or more peptide sequences or at the appropriate place, where amino acids are identical or possess similar chemical and/or physical properties such as charge or hydrophobicity. A so-termed “percent similarity” then can be determined between the compared peptide sequences. In general, “identity” refers to an exact amino acid to amino acid correspondence of two peptide sequences.

Two or more peptide or protein sequences can also be compared by determining their “percent identity”. The percent identity of two sequences may be described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). Suitable programs for calculating the percent identity or similarity between sequences are generally known in the art.

Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

In an embodiment, the signal peptidase sequence comprises, consists or consists essentially of an amino acid sequence selected from the group consisting of SEQ ID NOs:17-24.

The polyproteins encoded by the nucleic acid constructs disclosed herein will suitably comprise the two or more viral structural protein and the signal peptidase sequence(s) in a linear configuration, including as a fusion protein. As used herein, the term “fusion protein” typically refers to a polypeptide or polyprotein composed of two or more peptide or protein sequences linked to one another. In an embodiment, the encoded polyprotein comprises two or more viral structural protein sequences linked to the signal peptidase sequence(s) end-to-end. In an embodiment, each of the two or more viral structural proteins of the polyprotein are separated by a signal peptidase sequence. Illustrative examples of suitable polyprotein configurations are described below, where S is a viral structural protein and SP is signal peptidase sequence:
S1-SP1-S2
S1-SP1-S2-SP1-S3
S1-SP1-S2-SP2-S3
S1-SP1-S2-SP1-S3-SP1-S4
S1-SP1-S2-SP2-S3-SP2-S4
S1-SP1-S2-SP1-S3-SP2-S4
S1-SP1-S2-SP2-S3-SP3-S4

It is to be understood that any suitable configuration may be used, as long as any two of the viral structural protein sequence are separated by a signal peptidase sequence, as described herein.

In some embodiments, each of the two or more viral structural proteins of the polyprotein may be separated from one another by a signal peptidase sequence, as noted elsewhere herein, such that each of the viral structural proteins are liberated by host cell peptidase-dependent cleavage of the polyprotein. However, it is to be understood that the polyprotein may suitably comprise two or more viral structural proteins that are not separated by a signal peptidase sequence, but the polyprotein is otherwise capable of generating a functional VLP following host cell peptidase-dependent cleavage of the polyprotein. Illustrative examples of suitable polyprotein configurations of this type, wherein at least two of the two or more viral structural proteins are separated by a signal peptidase sequence, are described below, where S is a viral structural protein sequence and SP is a signal peptidase sequence:
S1-SP1-S2-S3
S1-S2-SP1-S3
S1-SP1-S2-S3-SP2-S4
S1-SP1-S2-S3-SP1-S4
S1-SP1-S2-SP2-S3-S4
S1-S2-SP1-S3-SP2-S4
S1-S2-SP1-S3-S4

In an embodiment, where the polyprotein comprises three viral structural proteins, two or more of the viral structural proteins of the polyprotein are separated from one another by a signal peptidase sequence. In an embodiment, where the polypeptide comprises three viral structural proteins, each of the three viral structural proteins of the polyprotein are separated from one another by a signal peptidase sequence. In an embodiment, where the polyprotein comprises four viral structural proteins, two or more of the viral structural proteins of the polyprotein are separated from one another by a signal peptidase sequence. In an embodiment, where the polyprotein comprises four viral structural proteins, each of the four viral structural proteins of the polyprotein are separated from one another by a signal peptidase sequence. In an embodiment, where the polyprotein comprises five viral structural proteins, two or more of the viral structural proteins of the polyprotein are separated from one another by a signal peptidase sequence. In an embodiment, where the polyprotein comprises five viral structural proteins, each of the five viral structural proteins of the polyprotein are separated from one another by a signal peptidase sequence. In an embodiment, where the polyprotein comprises six viral structural proteins, two or more of the viral structural proteins of the polyprotein are separated from one another by a signal peptidase sequence. In an embodiment, where the polyprotein comprises six viral structural proteins, each of the six viral structural proteins of the polyprotein are separated from one another by a signal peptidase sequence. In an embodiment, the two or more viral structural protein sequences of the polyprotein are linked to one or two signal peptidase sequences via a suitable linking moiety, also referred to herein as a linker. Suitable methods of linking peptide sequences will be familiar to persons skilled in the art, illustrative examples of which include peptide (amide) bonds and linkers. As used herein, the term “linker” refers to a short polypeptide sequence interposed between any two neighboring peptide or protein sequences as herein described. In an embodiment, the linker is a polypeptide linker of 1 to 10 amino acids, preferably 1, 2, 3, 4 or 5 naturally or non-naturally occurring amino acids. In an embodiment, the linker is a carbohydrate linker. Suitable carbohydrate linkers will be known to persons skilled in the art. In another embodiment disclosed herein, the fusion protein comprises one or more peptidic or polypeptidic linker(s) together with one or more other non-peptidic or non-polypeptidic linker(s). Further, different types of linkers, peptidic or non-peptidic, may be incorporated in the same fusion peptide as deemed appropriate. In the event that a peptidic or polypeptidic linker is used to join two respective peptide sequences, the linker will be advantageously incorporated such that its N-terminal end is bound via a peptide bond to the C-terminal end of the one peptide sequence, and its C-terminal end via a peptide bond to the N-terminal end of the other peptide sequence. The individual peptide sequences within the fusion protein may also have one or more amino acids added to either or both ends, preferably to the C-terminal end. Thus, for example, linker or spacer amino acids may be added to the N- or C-terminus of the peptides or both, to link the peptides and to allow for convenient coupling of the peptides to each other and/or to a delivery system such as a carrier molecule serving as an anchor. An illustrative example of a suitable peptidic linker is LP (leucine-proline).

As noted elsewhere herein, the present inventor has surprisingly found that, by employing two or more constructs encoding an immunogen and a polyprotein comprising viral structural proteins, as described herein, there is an unexpected improvement in VLP yield when the constructs are co-expressed in a host cell. Thus, the present disclosure extends to a system or composition comprising (i) a first construct comprising a nucleic acid sequence encoding an immunogen, wherein the immunogen comprises a B cell epitope and/or a T cell epitope of a SARS-CoV surface protein; and (ii) a second construct comprising a nucleic acid sequence encoding a polyprotein, wherein the polyprotein comprises two or more viral structural proteins, wherein at least two of the two or more viral structural proteins are separated by a signal peptidase sequence such that, when the polyprotein and the immunogen are expressed in a host cell, the signal peptidase sequence of the polyprotein undergoes host cell peptidase-dependent cleavage to liberate the two or more viral structural proteins, thereby allowing the liberated structural proteins and the immunogen to self-assemble into a VLP.

In an embodiment, the SARS-CoV surface protein is a SARS-CoV spike protein. In an embodiment, the SARS-CoV spike protein is a SARS-CoV-2 spike protein. Suitable SARS-CoV spike proteins will be familiar to persons skilled in the art, illustrative examples of which are described elsewhere herein. By employing a first construct encoding the immunogen and a second construct encoding the polyprotein of viral structural proteins, the system or composition described herein advantageously allows the first construct to be modified to encode a B cell epitope and/or a T cell epitope of the SARS-CoV surface protein having a sequence derived from a new or emerging variant of SARS-CoV, without having to modify the second construct. In other words, the system or composition described herein can be modified or adapted for raising an immune response against a new or emerging variants of SARS-CoV (including SARS-CoV-2 variants) without having to modify the second construct encoding the polyprotein.

In some embodiments, the first construct may further comprise a nucleic acid sequence encoding at least one viral structural protein, wherein the immunogen and the at least one viral structural protein are separated by a signal peptidase sequence, such that, when the polyprotein of the first construct is expressed in a host cell, the signal peptidase sequence undergoes host cell peptidase-dependent cleavage to liberate the immunogen and the at least one viral structural protein, the immunogen and viral structural proteins encoded by the first and second constructs, once liberated by host cell peptidase-dependent cleavage of the signal peptidase sequence(s), are capable of self-assembling to form a VLP. The term “system”, as used herein, typically refers to the first and second constructs as separate entities, permitting the first and second constructs to be introduced separately into a host cell. This advantageously allows the user to substitute, for example, the first construct with another first construct encoding a different immunogen whilst deploying the same second construct encoding the self-cleaving polyprotein of viral structural proteins for self-assembly with the immunogen to form the VLP.

In another aspect disclosed herein, there is provided a method of producing a VLP, the method comprising:
(i) introducing the first and second vaccine constructs, as described herein, into a host cell; and
(ii) culturing the host cell of (i) under conditions and for a period of time sufficient for the host cell to produce the VLP.

The present disclosure also extends to a VLP produced by the methods described herein, host cells and vaccine compositions comprising the vaccine constructs or the VLP, as described herein.

Suitable methods for preparing a nucleic acid sequence encoding the polyproteins disclosed herein will be familiar to persons skilled in the art, based on knowledge of the genetic code, possibly including optimising codons based on the nature of the host cell (e.g. microorganism) to be used for expressing and/or secreting the recombinant immunogen. Suitable host cells will also be known to persons skilled in the art, illustrative examples of which are described elsewhere herein and include prokaryotic cells (e.g., E. coli) and eukaryotic cells (e.g., P. pastoris). Reference is made to “Short Protocols in Molecular Biology, 5th Edition, 2 Volume Set: A Compendium of Methods from Current Protocols in Molecular Biology” (by Frederick M. Ausubel (author, editor), Roger Brent (editor), Robert E. Kingston (editor), David D. Moore (editor), J. G. Seidman (editor), John A. Smith (editor), Kevin Struhl (editor), J Wiley & Sons, London).

As used herein, the terms “encode,” “encoding” and the like refer to the capacity of a nucleic acid to provide for another nucleic acid or a polypeptide or polyprotein. For example, a nucleic acid sequence is said to “encode” a polypeptide or polyprotein if it can be transcribed and/or translated, typically in a host cell, to produce the polypeptide or polyprotein or if it can be processed into a form that can be transcribed and/or translated to produce the polypeptide or polyprotein. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product. In some embodiments, the nucleic acid sequence encoding the immunogens, the viral structural proteins and/or the signal peptidase sequences, as herein described, are codon-optimized for expression in a suitable host cell. For example, where the VLP are to be used for inducing an immune response in a human subject, the nucleic acid sequences encoding the immunogens, the viral structural proteins and/or the signal peptidase sequences, as herein described, can be human codon-optimized. Suitable methods for codon optimization would be known to persons skilled in the art, such as using the “Reverse Translation” option of ‘Gene Design” tool located in “Software Tools” on the John Hopkins University Build a Genome website.

### Host Cells

In another aspect disclosed herein, there is provided a host cell comprising the nucleic acid construct, as described herein. In an embodiment, the host cell is permissive for viruses. Suitable host cells permissive for viruses will be familiar to persons skilled in the art, illustrative examples of which are described in Sarkar et al. (Korean J. Microbiol. 2019; 55(4):327-343), Santi et al. (Methods. 2006; 40(1): 66-76), Roldio et al. (Expert Rev. Vaccines; 2010; 9(10): 1149-1176) and (Ghislain Masavuli et al. Front Microbiol. 2017; 8: 2413), and include bacteria, yeast, fungi, plant, insect, mammalian and avian cells.

In an embodiment, the host cell is a bacterium. Suitable virus permissive bacteria will be familiar to persons skilled in the art, an illustrative example of which includes Escherichia coli (Huang et al., NPJ Vaccines. 2017; 2:3).

In an embodiment, the host cell is a yeast cell. Suitable virus permissive yeast cells will be familiar to persons skilled in the art, illustrative examples of which are described in Kumar and Kumar (FEMS Yeast Research, 2019; 19(2): foz007) and include Pichia Pastoris (Kim and Kim, Letters Appl. Micro., 2017; 64(2):111-123), Saccharomyces cerevisiae (Zhao et al. Appl Microbiol Biotechnol. 2013; 97(24):10445-52) and Hansenula polymorpha (Wetzel et al. J Biotechnol. 2019; 20; 306:203-212). In an embodiment, the host cell is Pichia Pastoris. In an embodiment, the host cell is Saccharomyces cerevisiae. In an embodiment, the host cell is Hansenula polymorpha.

In an embodiment, the host cell is a plant cell. Suitable virus permissive plant cells will be familiar to persons skilled in the art, illustrative examples of which are described in Makarkov et al. (npj Vaccines; 2019; 4(17)) and Santi et al. (Methods. 2006; 40(1): 66-76), and include Nicotiana tabacum and Arabidopsis thaliana (Greco et al. Vaccine. 2007; 28; 25(49):8228-40), Samsun NN and Solanum tuberosum cv. Solara. In an embodiment, the host cell is Trichoplusia ni (BTI-TN-5B1-4). In an embodiment, the host cell is Nicotiana tabacum cv. Samsun NN. In an embodiment, the host cell is Solanum tuberosum cv. Solara.

In an embodiment, the host cell is an insect cell. Suitable virus permissive insect cells will be familiar to persons skilled in the art, illustrative examples of which include Spodoptera frugiperda (sf9; Wagner et al. PLoS One. 2014; 9(4):e94401), Trichoplusia ni (BTI-TN5B1-4; Krammer et al., Mol Biotechnol. 2010; 45(3):226-234) and Drosophila Schneider 2 (S2; Park et al., J Virol Methods. 2018; 261:156-159). In an embodiment, the host cell is Spodoptera frugiperda. In an embodiment, the host cell is Trichoplusia ni. In an embodiment, the host cell is Drosophila Schneider 2.

In an embodiment, the VLP is produced in a mammalian cell. Mammalian cells are used in expression of various proteins because of their ability to carry out the post translational modifications (PTM). An additional advantage of this system is that the proteins are secreted in their native, mature form. Suitable mammalian host cells will be familiar to persons skilled in the art, illustrative examples of which include Chinese hamster ovary (CHO) cells (Michel et al., 1984, Proc. Natl. Acad. Sci. USA. 81, 7708-7712; Patzer et al. 1986, J. Virol. 58, 884-892); Purdy and Chang, 2005, Virology. 333, 239-250), Vero cells, Human Embryonic Kidney 293 cells (Buffin et al., Vaccine, 2019; 37(46): 6857-6867) and Huh7 human liver cells (Earnest-Silveira et al., J Gen Virol. 2016; 97(8):1865-1876). In an embodiment, the host cell is Chinese hamster ovary (CHO). In an embodiment, the host cell is Human Embryonic Kidney 293. In an embodiment, the host cell is a Huh7 cell.

### VLP Production

The present disclosure also extends to methods of manufacturing the VLP described herein. Thus, in an aspect disclosed herein, there is provided a method of producing a modified virus-like particle (VLP), the method comprising:
(i) introducing the nucleic acid construct as described herein into a host cell; and
(ii) culturing the host cell of (i) under conditions and for a period of time sufficient for the host cell to produce the VLP.

Suitable culture methods and conditions/times suitable for producing VLP will be familiar to persons skilled in the art, understanding that the method steps and conditions/times will likely vary depending, for example, on the type of host cell employed. Illustrative examples of suitable methods of production are set out elsewhere herein and also described in Cheng and Mukhopadhyay (Virology, 2011; 413(2):153-160), Charlton Hume et al. (ibid), Ghislain Masavuli et al. (ibid) and Vacher et al. (Mol. Pharmaceutics 2013; 10:1596-1609).

Nucleic acid sequences encoding the polyprotein, as described herein, can be readily prepared by standard genetic engineering techniques by the skilled person provided with the sequence of the SARS-CoV protein or genomic sequence. Methods of genetically engineering the nucleic acid constructs described herein are well known in the art, illustrative examples of which are described, for example, in Ausubel et al. (1994 and updates) Current Protocols in Molecular Biology, John Wiley & Sons, New York).

Isolation and cloning of the nucleic acid sequence encoding the polyprotein described herein can be achieved using standard techniques (see, e.g., Ausubel et al., ibid.). For example, the nucleic acid sequence can be obtained directly from the SARS-CoV virus by extracting RNA by standard techniques and then synthesizing cDNA from the RNA template (e.g., by RT-PCR). The nucleic acid sequence encoding the virus structural protein(s) is then inserted directly or after one or more subcloning steps into a suitable expression vector. Persons skilled in the art will understand that the precise vector used is not critical. Illustrative examples of suitable vectors include plasmids, phagemids, cosmids, bacteriophage, baculoviruses, retroviruses or DNA viruses. The viral structural protein(s) can then be expressed and purified as described in more detail below. Alternatively, the nucleic acid sequence encoding the viral structural protein(s) can be further engineered to introduce one or more mutations, such as those described above, by standard in vitro site-directed mutagenesis techniques known to persons skilled in the art. Mutations can be introduced by deletion, insertion, substitution, inversion, or a combination thereof, of one or more of the appropriate nucleotides making up the coding sequence. This can be achieved, for example, by PCR based techniques for which primers are designed that incorporate one or more nucleotide mismatches, insertions or deletions. The presence of the mutation can be verified by a number of standard techniques, for example by restriction analysis or by DNA sequencing.

Virus proteins may also be engineered to produce fusion proteins comprising one or more immunogens fused to virus coat protein. Methods for making fusion proteins are well known to those skilled in the art. DNA sequences encoding a fusion protein can be inserted into a suitable expression vector as noted above. Persons skilled in the art will appreciate that the DNA encoding the coat protein or fusion protein can be altered in various ways without affecting the activity of the encoded protein. For example, variations in DNA sequence may be used to optimize for codon preference in a host cell used to express the protein, or may contain other sequence changes that facilitate expression.

It will be understood that the expression vector may further include regulatory elements, such as transcriptional elements, required for efficient transcription of the DNA sequence encoding the coat or fusion protein. Illustrative examples of suitable regulatory elements that can be incorporated into the vector include promoters, enhancers, terminators, and polyadenylation signals. The present disclosure therefore also provides vectors comprising a regulatory element operatively linked to one or more nucleic acid sequences encoding the VLP described herein. Persons skilled in the art will appreciate that selection of suitable regulatory elements may be dependent on the host cell chosen for expression of the VLP and that such regulatory elements may be derived from a variety of sources, including bacterial, fungal, viral, plant, mammalian or insect genes.

The expression vector may additionally contain heterologous nucleic acid sequences that facilitate the purification of the expressed VLP. Illustrative examples of suitable heterologous nucleic acid sequences include affinity tags such as metal-affinity tags, histidine tags, avidin/streptavidin encoding sequences, glutathione-S-transferase (GST) encoding sequences and biotin encoding sequences. The amino acids corresponding to expression of the nucleic acids can be removed from the expressed VLP prior to use, including clinical use, as described elsewhere herein. Alternatively, the amino acids corresponding to expression of heterologous nucleic acid sequences can be retained on the VLP if they do not interfere with subsequent use.

The expression vector can be introduced into a suitable host cell by one of a variety of methods known in the art, illustrative examples of which are generally described in Ausubel et al. (ibid.) and include, for example, stable or transient transfection, lipofection, electroporation, and infection with recombinant viral vectors. One skilled in the art will understand that selection of the appropriate host cell for expression of the coat protein will be dependent upon the vector chosen. Illustrative examples of suitable host cells are described elsewhere herein and include bacterial, yeast, insect, plant and mammalian cells.

It is to be understood that the recombinant viral structural proteins should be capable of multimerization and assembly into VLP. In general, assembly takes place in the host cell expressing the structural proteins. The VLP can be isolated from the host cells by standard techniques known to persons skilled in the art, such as those described elsewhere herein. The VLP can be further purified by standard techniques, such as chromatography, to remove contaminating host cell proteins or other compounds.

Methods for introducing the nucleic acid construct into a host cell for the purposes of producing VLP will be known to persons skilled in the art, illustrative examples of which include transformation, transduction, electroporation, conjugation, transfection, calcium phosphate methods, and the like.

Host cells expressing one or more of the sequences described herein can readily be generated given the disclosure provided herein by stably integrating the sequences into the genome of a host cell, to allow for the formation of the VLP, as described herein. The promoter regulating expression of the stably integrated nucleic acid sequences(s) may be constitutive or inducible. Thus, a host cell can be generated in which the VLP proteins are stably integrated such that, upon introduction of the nucleic acid-encoding sequences or expression construct comprising these virus sequences into the host cell of the proteins encoded by said nucleic acid sequences, form non-replicating VLP.

When the genes that code for the proteins required for VLP formation are introduced into a host cell and subsequently expressed at the necessary level, the VLP assembles and can then be released from the cell into the culture media, where it can be further processed (e.g., purified), if necessary, having regard to the intended use.

Depending on the expression system and host cell selected, the VLP, as herein defined, are typically produced by growing the host cell transformed by the expression vector under conditions whereby the viral proteins encoded by the construct/expression vector are expressed and VLP can be formed. The selection of the appropriate growth conditions will be familiar to persons skilled in the art.

The VLP can be isolated (or substantially purified) using methods that preserve the integrity thereof, such as, by density gradient centrifugation, e.g., sucrose gradients, PEG-precipitation, pelleting, and the like (see, e.g., Kirnbauer et al. J. Virol. (1993) 67:6929-6936), as well as standard purification techniques including, e.g., ion exchange and gel filtration chromatography. For example, a composition or preparation comprising an isolated VLP prepared according to the method of the present disclosure may comprise at least 65%, at least 70%, at least 75%, at least 80%, at least 85%, at least 90%, at least 95%, at least 96%, at least 98%, at least 99% or 100% of an isolated VLP, as measured by methods known to persons skilled in the art.

The presence of the VLP can be detected using conventional techniques known in the art, such as by electron microscopy, atomic force microscopy, biophysical characterization, and the like (see, e.g., Baker et al., Biophys. J. (1991) 60:1445-1456; and Hagensee et al., J. Virol. (1994) 68:4503-4505). For example, the VLP can be isolated by density gradient centrifugation and/or identified by characteristic density banding. Alternatively, cryoelectron microscopy can be performed on vitrified aqueous samples of the VLP preparation in question, and images recorded under appropriate exposure conditions.

The present disclosure also extends to a VLP produced by the methods described herein and a vaccine composition comprising the nucleic acid constructs and/or VLP, as described herein.

### Methods of Treatment

The nucleic acid constructs, VLP and compositions described herein may be used to potentiate an immune response in an animal, or as vaccines to induce a protective or therapeutic immune response to a SARS-CoV in a host. Thus, in another aspect disclosed herein, there is provided a method of raising an immune response in a subject to a SARS-CoV, the method comprising administering to a subject in need thereof the nucleic acid construct, VLP or composition described herein. In another aspect disclosed herein, there is provided use of the nucleic acid construct, VLP or composition described herein, in the manufacture of a medicament for raising an immune response in a subject to the SARS-CoV. In an embodiment, the immune response generates antibodies to the immunogen. In yet another aspect disclosed herein, there is provided the nucleic acid construct, VLP or composition described herein for use in raising an immune response in a subject against a SARS-CoV. In an embodiment, the nucleic acid construct, VLP or composition described herein induces a humoral, cellular and/or innate immune response in the subject to which it is administered.

The nucleic acid constructs or VLP described herein may be advantageously formulated as pharmaceutical compositions together with a pharmaceutically acceptable carrier, diluent, and/or excipient. Suitable carriers, diluents, and/or excipients are well known in the art. If desired, an adjuvant or other active ingredient optionally may be included in the compositions. In an embodiment, the compositions are capable of efficiently potentiating an immune response in the absence of an additional adjuvant.

For administration to a host, the compositions described herein can be formulated for administration by a variety of routes. For example, the compositions can be formulated for oral, topical, rectal or parenteral administration or for administration by inhalation, intranasally or spray. The term “parenteral”, as used herein, includes subcutaneous injections, intradermal, intravenous, intramuscular, intrathecal, intrasternal injection and infusion techniques. In an embodiment, the compositions are formulated for topical, rectal or parenteral administration or for administration by inhalation, intranasally or spray. In an embodiment, the compositions are formulated for parenteral administration.

The compositions described herein will suitably comprise a therapeutically effective amount of the nucleic acid construct or VLP. The phrase “therapeutically effective amount” typically means an amount of the vaccine construct and/or VLP, as described herein, necessary to attain the desired response, for example, the inducement of an immune response to the SARS-CoV. Typically, the appropriate dosage of the nucleic acid construct and/or VLP, as described herein, may depend on a variety of factors including, but not limited to, a subject's physical characteristics (e.g., age, weight, sex), whether the nucleic acid construct and/or VLP, as described herein, is being used as single agent or as part of adjuvant therapy, the progression (i.e., pathological state) of any underlying virus infection or disease, and other factors that may be recognized by persons skilled in the art. Various general considerations that may be considered when determining, for example, an appropriate dosage of the vaccine composition (see, e.g., in Gennaro (2000) “Remington: The Science and Practice of Pharmacy”, 20th edition, Lippincott, Williams, & Wilkins; and Gilman et al., (Eds), (1990), “Goodman And Gilman's: The Pharmacological Bases of Therapeutics”, Pergamon Press). It is expected that the amount will fall in a relatively broad range that can be determined through methods known to persons skilled in the art. Illustrative examples of a suitable therapeutically effective amount of VLP for administration to a human subject include from about 0.001 mg per kg of body weight to about 1 g per kg of body weight, preferably from about 0.001 mg per kg of body weight to about 50 g per kg of body weight, more preferably from about 0.01 mg per kg of body weight to about 1.0 mg per kg of body weight. Dosage regimes may be adjusted to provide the optimum therapeutic response. For example, several divided doses may be administered daily, weekly, monthly or other suitable time intervals, or the dose may be proportionally reduced as indicated by the exigencies of the situation.

In an embodiment, a therapeutically effective amount of the nucleic acid construct and/or VLP, as described herein, as defined herein, is effective to induce an immune response to the SARS-CoV, irrespective of the genotype.

As described elsewhere herein, the terms “immune response”, “immunological response” and the like are typically used herein to refer to the development in a subject of a humoral and/or a cellular immune response to the SARS-CoV. A “humoral immune response” typically refers to an immune response mediated by antibody molecules, while a “cellular immune response” is typically mediated by SARS-CoV-specific T-lymphocytes and/or other white blood cells. In a non-limiting example, the vaccine construct and/or VLP, as described herein, when administered to a subject induces an immune response selected from one or more of a neutralising antibody response, a cytotoxic T lymphocyte (CTL) response, a natural killer T cell response and/or a helper T lymphocyte (e.g., CD4+ T cell) response and innate immune response to the SARS-CoV.

Methods for measuring an immune response will be known to persons skilled in the art, illustrative examples of which include plaque-reduction neutralization assay, micro-neutralization assay, solid-phase heterogeneous assays (e.g., enzyme-linked immunosorbent assay), solution phase assays (e.g., electrochemiluminescence assay), amplified luminescent proximity homogeneous assays, flow cytometry, intracellular cytokine staining, functional T-cell assays including suppressor T-cell assays, functional B-cell assays, functional monocyte-macrophage assays, dendritic and reticular endothelial cell assays, measurement of NK or NKT cell responses, oxidative burst assays, cytotoxic-specific cell lysis assays, pentamer binding assays, and phagocytosis and apoptosis evaluation.

As described elsewhere herein, the nucleic acid construct and/or VLP, as described herein, can be administered to a subject in need thereof by any suitable route of administration, including administration of the composition orally, nasally, nasopharyngeally, parenterally, enterically, gastrically, topically, transdermally, subcutaneously, intramuscularly, intradermally, in tablet, solid, powdered, liquid, aerosol form, locally or systemically, with or without added excipients. Suitable methods for preparing parenterally administrable compositions will be known or apparent to those skilled in the art and are described in more detail in such publications as Remington's Pharmaceutical Science, 15th ed., Mack Publishing Company, Easton, Pa. (1980). In some instances, administration is accomplished by intramuscular injection of the nucleic acid construct and/or VLP. In general, the nucleic acid construct and/or VLP, as described herein, can be administered in a manner compatible with the route of administration and physical characteristics of the recipient (including health status) and in such a way that it elicits the desired effect(s) (e.g. the induction of a protective immune response against the SARS-CoV).

The nucleic acid construct and/or VLP, as described herein, may be administered to a recipient in isolation or in combination with other additional therapeutic agent(s). In embodiments where a pharmaceutical composition comprising the nucleic acid construct and/or VLP, as described herein, is formulated for administration with additional therapeutic agent(s), the administration may be simultaneous or sequential (i.e., administration of the nucleic acid construct and/or VLP is followed by administration of the additional agent(s) or vice versa). Thus, here two or more entities are administered to a subject “in conjunction”, they may be administered in a single composition at the same time, or in separate compositions at the same time, or in separate compositions separated in time.

In a non-limiting example, the nucleic acid construct and/or VLP, as described herein, may be administered in conjunction with an antiviral agent. An “antiviral agent or compound” is defined as an agent which, when administered to a subject, is capable of significantly reducing the virus titer in the blood or serum either directly (e.g., by inhibiting a viral enzyme activity) or indirectly (e.g., via modulation of the antiviral responses of a host cell), either transiently or in a sustained way. In general, the term antiviral agents comprise any pharmaceutically acceptable form of said agents including pharmaceutically acceptable salts and solvents as long as the biological effectiveness of the antiviral agent is not significantly compromised. Suitable antiviral agents would be known to persons skilled in the art, illustrative examples of which include sofosbuvir, ledipasvir, ribavirin, paritaprevir, and simeprevir.

In an embodiment, the immune response that is raised by the methods, uses and compositions described herein comprises an innate immune response. In an embodiment, the innate immune response comprises activation of NKT cells. In an embodiment, the immune response further comprises an adaptive immune response. In an embodiment, the adaptive immune response comprises activation of adaptive CD4 and/or CD8 T lymphocytes.

Various pharmaceutical compositions suitable for different routes of administration and methods of preparing pharmaceutical compositions will be known in the art, illustrative examples of which are described in “Remington: The Science and Practice of Pharmacy” (formerly “Remingtons Pharmaceutical Sciences”); Gennaro, A., Lippincott, Williams & Wilkins, Philadelphia, Pa. (2000).

In an embodiment, the nucleic acid construct and/or VLP, as described herein, may suitably be given in an appropriate single dosage in order to elicit an immune response. In other embodiments, the initial dose may be followed by boosting dose. The boosting dose may comprise the same nucleic acid construct and/or VLP as the initial (priming) dose, whether at an equivalent dose (e.g., the same or similar dose), a lower dose or a higher dose as compared to the initial dose, or it may comprise the immunogen alone (e.g., the viral structural antigen, such as the SARS-CoV spike protein or an immunogenic fragment thereof), or the immunogen in combination with a suitable adjuvant, as described elsewhere herein; that is, in the absence of the nucleic acid construct and/or VLP.

The administration regime need not differ from any other generally accepted vaccination programs. For instance, a single administration in an amount sufficient to elicit an effective immune response may be used. Alternatively, as noted above, other regimes of initial administration of the complex followed by boosting with immunogen alone, including as described above. Boosting may occur at times that take place well after the initial administration if the immune response (as measured, e.g., by antibody titres) falls below acceptable levels.

Alternatively, or in addition, the nucleic acid construct and/or VLP, as described herein, can be used in combination an additional immunopotentiator or adjuvant to enhance an immune response in humans or non-human animals against the targeted antigen(s). The present disclosure therefore extends to compositions further comprising an immunopotentiator or adjuvant. Preferably, the immunopotentiator or adjuvant is administered concomitantly with the nucleic acid construct and/or VLP, as described herein. The immunopotentiator or adjuvant can be administered prior or subsequently to the nucleic acid construct and/or VLP, as described herein, depending on the need as can be suitably determined by persons skilled in the art. The term “immunopotentiator,” as used herein, is intended to mean a substance that, when mixed with an immunogen, elicits a greater immune response than the immunogen alone. For example, an immunopotentiator can enhance immunogenicity and provide a superior immune response. An immunopotentiator can act, for example, by enhancing the expression of co-stimulators on macrophages and other antigen-presenting cells.

Suitable immunopotentiators or adjuvants will be familiar to persons skilled in the art, illustrative examples of which include 1018 ISS, aluminum salts, AMPLIVAX®, AS15, BCG, CP-870,893, CpG7909, CyaA, dSLIM, flagellin or TLRS ligands derived from flagellin, FLT3 ligand, GM-CSF, IC30, IC31, Imiquimod (ALDARA™), resiquimod, ImuFact IMP321, Interleukins such as IL-2, IL-12, IL-18, IL-21, Interferon-alpha or -beta or -gamma or pegylated derivatives thereof, IS Patch, ISS, ISCOMATRIX, ISCOMs, JuvImmune™, LipoVac, MALP2, MF59, monophosphoryl lipid A, Montanide IMS 1312, Montanide ISA 206, Montanide ISA 50V, Montanide ISA-51, other suitable water-in-oil and oil-in-water emulsions (e.g., AddaVax), OK-432, OM-174, OM-197-MP-EC, ONTAK, OspA, PepTel™. vector system, poly(lactid co-glycolid) [PLG]-based and dextran microparticles, talactoferrin SRL172, YF-17D, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, Quil, or Superfos. Adjuvants such as Freund's or GM-CSF are preferred. Several immunological adjuvants (e.g., MF59) specific for dendritic cells and their preparation have been described previously (Allison and Krummel, 1995, Journal of Experimental Medicine 182(2):459). Immunopotentiating cytokines may also be used. Several cytokines have been directly linked to influencing dendritic cell migration to lymphoid tissues (e.g., TNF-), accelerating the maturation of dendritic cells into efficient antigen-presenting cells for T-lymphocytes (e.g., GM-CSF, IL-1 and IL-4) (see, e.g., U.S. Pat. No. 5,849,589, specifically incorporated herein by reference in its entirety) and acting as immunoadjuvants (e.g., IL-12, IL-15, IL-23, IL-7, IL-18, IFN-alpha. IFN-beta).

CpG immunostimulatory oligonucleotides have also been reported to enhance the effects of adjuvants in a vaccine setting. Without being bound by theory or by a mode of application, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly, it can enhance dendritic cell maturation and differentiation, resulting in enhanced activation of TH1 cells and strong cytotoxic T-lymphocyte (CTL) generation, even in the absence of CD4 T cell help. The TH1 bias induced by TLR9 stimulation is maintained even in the presence of vaccine adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nanoparticles, lipid emulsions or similar formulations, which are especially necessary for inducing a strong response when the antigen is relatively weak. They also accelerate the immune response and enable the antigen doses to be reduced by approximately two orders of magnitude, with comparable antibody responses to the full-dose vaccine without CpG. U.S. Pat. No. 6,406,705 describes the combined use of CpG oligonucleotides, non-nucleic acid adjuvants and an antigen to induce an antigen-specific immune response. A CpG TLR9 antagonist is dSLIM (double Stem Loop Immunomodulator) by Mologen (Berlin, Germany) which is a preferred component of the pharmaceutical composition of the present invention. Other TLR binding molecules such as RNA binding TLR 7, TLR 8 and/or TLR 9 may also be used.

Other illustrative examples of suitable adjuvants include chemically modified CpGs (e.g. CpR, Idera), dsRNA analogues such as Poly(I:C) and derivates thereof (e.g. AmpliGen®, Hiltonol®, poly-(ICLC), poly(IC-R), poly(I:C12U), non-CpG bacterial DNA or RNA as well as immunoactive small molecules and antibodies such as cyclophosphamide, sunitinib, Bevacizumab®, celebrex, NCX-4016, sildenafil, tadalafil, vardenafil, sorafenib, temozolomide, temsirolimus, XL-999, CP-547632, pazopanib, VEGF Trap, ZD2171, AZD2171, anti-CTLA4, other antibodies targeting key structures of the immune system (e.g. anti-CD40, anti-TGFbeta, anti-TNF receptor) and SC58175, which may act therapeutically and/or as an adjuvant. The amounts and concentrations of adjuvants and additives useful in the context of the present invention can readily be determined by the skilled artisan without undue experimentation.

In an embodiment, the adjuvant is selected from the group consisting of anti-CD40, imiquimod, resiquimod, GM-CSF, cyclophosphamide, sunitinib, bevacizumab, interferon-alpha, CpG oligonucleotides and derivates, poly-(I:C) and derivates, RNA, sildenafil, and particulate formulations with PLG. In an embodiment, the adjuvant is an oil-in-water emulsion or an immunostimulatory lipid. In an embodiment, the adjuvant is an oil-in-water emulsion. In another embodiment, the adjuvant is a squalene-based, oil-in-water emulsion (e.g., AddaVax™).

In an embodiment, the compositions described herein further comprise an immunostimulatory or adjuvanting lipid. Suitable immunostimulatory lipids will be familiar to persons skilled in the art, illustrative examples of which include glycolipids and phospholipids. In an embodiment, the immunostimulatory lipid is an immunostimulatory glycolipid or an immunostimulatory phospholipid. The immunostimulatory lipid may be a naturally-occurring lipid or it may be synthetically derived (synthesized). In some embodiments, the immunostimulatory lipid is a synthetic lipid. In some embodiments, the synthetic lipid may resemble, in part or in whole, a naturally-occurring lipid. Thus, also contemplated herein are immunostimulatory lipid analogues; that is, synthetic immunostimulatory lipids that resemble, at least in part, naturally-occurring lipids and yet are still capable of acting as an adjuvant to potentiate a host's immune response to an immunogen. Suitable immunostimulatory lipid analogues will be familiar to persons skilled in the art, illustrative examples of which are described in US patent publication nos. 20200009165 and 20190358318, Maeda et al. (Vaccine; 1989; 7(3):275-281); Jiang et al. (Carbohydr. Res., 2007; 342(6):784-796); Foster et al. (J. Med. Chem., 2018; 61(3):1045-1060), the contents of which are incorporated herein by reference in their entirety.

In an embodiment, the immunostimulatory lipid is a glycolipid. Suitable immunostimulatory glycolipids will be familiar to persons skilled in the art, illustrative examples of which are described in Kim et al. (Expert Rev Vaccines. 2008; 7(10): 1519-1532), Godfrey, et al. (Nat. Immunol 2015; 16: 1114-1123, and Cerundolo, et al. (Nat. Rev. Immunol. 2009; 9: 28-38). In an embodiment, the glycolipid is an alpha-anomeric glycolipid or a beta-anomeric glycolipid. In an embodiment, the glycolipid is an alpha-anomeric glycolipid. In an embodiment, the alpha-anomeric glycolipid is an alpha-anomeric glycosphingolipid. In an embodiment, the alpha-anomeric glycosphingolipid is α-galactosylceramide or α-glucosylceramide. In another embodiment, the alpha-anomeric glycosphingolipid is α-glucosylceramide. In another embodiment, the glycolipid is a beta-anomeric glycolipid. In another embodiment, the beta-anomeric glycolipid is a beta-anomeric glycosphingolipid. In another embodiment, the beta-anomeric glycolipid is selected from the group consisting of β-mannosylceramide, β-glucosylceramide and β-galactosylceramide. In another embodiment, the beta-anomeric glycolipid is β-mannosylceramide.

In another embodiment, the immunostimulatory lipid is a phospholipid. Suitable immunostimulatory phospholipids will be familiar to persons skilled in the art, illustrative examples of which are described in Godfrey, et al. (2015; ibid). In an embodiment, the phospholipid is lyso-phosphatidylcholine or lysophosphatidyl-ethanolamine. Immunostimulatory lipids, as described herein, including immunostimulatory glycolipids and phospholipids, can vary in the length and saturation of their fatty acid chains (including the acyl and/or sphingosine chains). These are typically referred to by reference to the number of carbons on the acyl chain (e.g., α-GalCer C26, α-GalCer C24, α-GalCer C20:2 etc). For instance, α-GalCer, also known as KRN7000, has an 18C phytosphingosine and a 26C acyl chain, whereas α-GalCer C20:2 has a C20 acyl chain and cis-diunsaturation at C11 and C14. Suitable immunostimulatory lipids of varying length and saturation of their fatty acid chains will be familiar to persons skilled in the art, illustrative examples are described in Wun et al. (2011. Immunity 34: 327-339). Such variation in the length and saturation of their fatty acid chains may impact on the immunostimulatory capacity of such lipids. Nevertheless, it is to be understood that the immunostimulatory lipids described herein, when administered to a host with an immunogen, will be capable of potentiating the host's immune response to the immunogen, irrespective of the length and saturation of their fatty acid chains. Methods of determining whether an immunostimulatory lipid is capable of potentiating the host's immune response to an immunogen according to the methods described herein, irrespective of the length and saturation of their fatty acid chains, will be known to persons skilled in the art.

In another embodiment, the immunostimulatory lipid is a natural killer T (NKT) cell agonist. In an embodiment, the immunostimulatory lipid is a type 1 and a type 2 NKT cell agonist. In an embodiment, the immunostimulatory lipid is a type 1 NKT cell agonist. In an embodiment, the immunostimulatory lipid is a type 2 NKT cell agonist. Type 1 and type 2 NKT cells are known in the art (see, e.g., Godfrey et al., 2015, ibid). Illustrative examples of suitable lipids that are capable of activating NKT cells are described in Kim et al. (ibid) and Godfrey et al. (2015, ibid), including those described elsewhere herein.

The terms “immunisation” and “vaccination” are used interchangeably herein to refer to the administration of the vaccine construct and/or VLP, as described herein, to a subject for the purposes of raising an immune response and can have a prophylactic effect, a therapeutic effect, or a combination thereof.

As used herein, the terms “treat,” “treated,” or “treating” when used with respect to a disease or pathogen refers to a treatment which increases the resistance of a subject to the disease (e.g., COVID-19) or to infection with SARS-CoV (i.e., decreases the likelihood that the subject will contract the disease or become infected with SARS-CoV), as well as a treatment after the subject has contracted the disease or become infected in order to fight a disease or infection (e.g., to reduce, eliminate, ameliorate or otherwise stabilise a disease or infection by the SARS-CoV). In an embodiment, the nucleic acid construct and/or VLP, as described herein, are capable of providing protective immunity to a host. The term “protective immunity,” as used herein, is intended to mean the ability of a host, such as a mammal, bird, or fish, to resist (delayed onset of symptoms, reduced severity of symptoms or lack of symptoms), as a result of its exposure to the nucleic acid construct and/or VLP, as described herein, disease or death that would otherwise follow exposure to a pathogen. Protective immunity is typically achieved by one or more of mucosal, humoral, or cellular immunity. Mucosal immunity is understood to mean the presence of secretory IgA (sIgA) antibodies on mucosal surfaces of the respiratory, gastrointestinal, and genitourinary tracts. Humoral immunity typically refers to the presence of IgG antibodies and IgM antibodies in serum. Cellular immunity typically refers to activation of cytotoxic T lymphocytes or delayed-type hypersensitivity that involves macrophages and T lymphocytes, as well as other mechanisms involving T cells without a requirement for antibodies.

The term “subject”, “host” or “patient” refers to an animal in need of treatment. The subject, host or patient encompasses human and non-human subjects, including, but not limited to, mammals, birds and fish, and suitably encompasses domestic, farm, zoo and wild animals, such as, for example, cows, pigs, horses, goats, sheep or other hoofed animals, dogs, cats, chickens, ducks, non-human primates, guinea pigs, rabbits, ferrets, rats, hamsters and mice. In an embodiment, the subject is a human.

The present disclosure also extends to pharmaceutical kits or packs comprising the nucleic acid construct, VLP and/or compositions, as described herein, including for use as a vaccine. Individual components of the kit can be packaged in separate containers, associated with which, when applicable, can be a notice in the form prescribed by a governmental agency regulating the manufacture, use or sale of pharmaceuticals or biological products, which notice reflects approval by the agency of manufacture, use or sale for human or animal administration.

The kits may optionally further comprise one or more other therapeutic agents for use in combination with the nucleic acid construct, VLP and/or compositions, as described herein. The kit may optionally contain instructions or directions outlining the method of use or administration regimen.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also suitably be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, syringe, pipette, forceps, measured spoon, eye dropper or similar medically approved delivery vehicle.

All patents, patent applications and publications mentioned herein are hereby incorporated by reference in their entireties.

Aspects disclosed herein are further described by the following non-limiting examples.

### EXAMPLES

### Example 1—Vaccine Construct Encoding a Self-Cleaving SARS-CoV-2 VLP Polyprotein

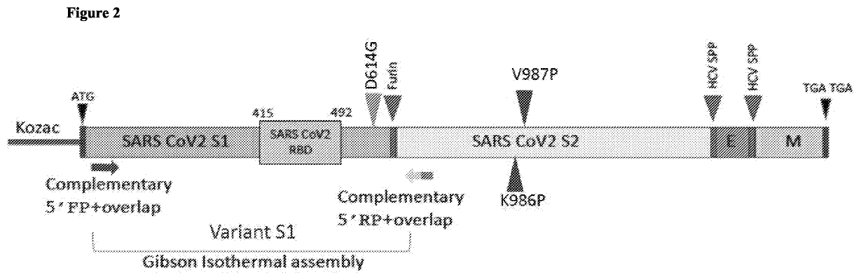

A nucleic acid construct was developed to produce a self-cleaving polyprotein comprising the spike (S), membrane (M) and envelope (E) proteins of SARS-CoV-2. A signalase sequence (SEQ ID NO:17) was introduced (i) between the S2 and E; and (ii) between the E and M proteins of SARS-CoV2, to facilitate self-cleavage of the expressed polyprotein and thereby liberate the S1, S2, E and M proteins that will then self-assembly into a SARS-CoV-2 VLP. The polyprotein also included a modified SARS-CoV-2 nucleoprotein (N) protein, which was found to improve the stability of the VLP. The RNA binding motifs of the N protein were mutated to ensure that the protein is not able to package RNA into the VLP. The vaccine constructs, when expressed in a host cell, produce VLP comprising SARS-CoV-2 S1, S2, E, M and (modified) N proteins. A diagrammatical representation of the SARS-CoV-2 polyprotein/vaccine construct is shown in FIG. 2.

To produce the SARS-CoV-2 nucleic acid construct, the genome sequence of the SARS-CoV-2 spike (S), envelope (E) and membrane (M) proteins were derived from GenBank Accession No. NC_045512.2, the entire contents of which is incorporated herein by reference.

The SARS-CoV-2 nucleic acid construct was synthesized by GeneArt and subcloned into an AdEasy expression system. The SARS-CoV-2 vaccine construct was then cloned into pAdTrack CMV to create the pAdTrack-CMV-SARS-CoV-2. This plasmid was enzymatically digested with PmeI and transformed into AdEasier cells by electroporation (Bio-Rad Gene Pulser). Positive clones were confirmed by restriction digestion with PacI, then transformed into Top 10 F′ cells.

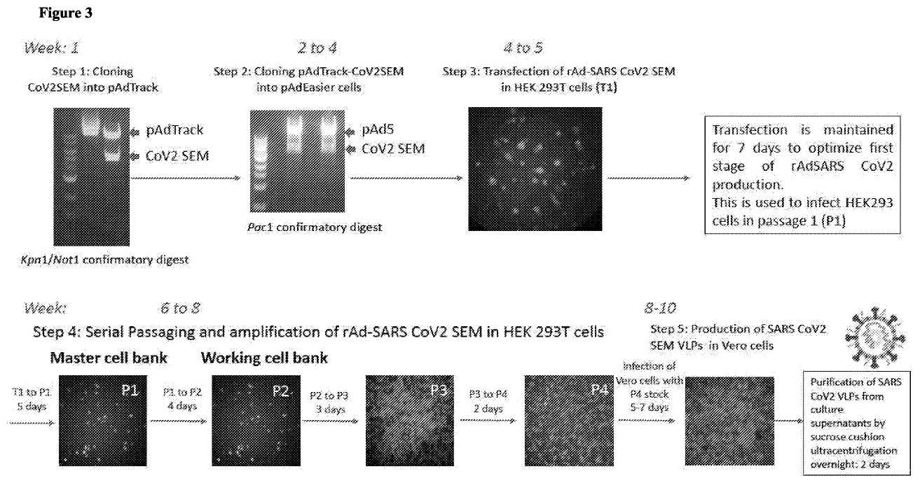

rAd-SARS-CoV-2-SEM viruses were produced by transfection of the generated plasmid DNA into 293T cells and high titres of recombinant adenoviruses encoding the structural proteins (rAd-SARS-CoV-2-S/E/M) were produced in 293T cells by serial passaging. Multiplicity of infection (MOI) was determined as described in Earnest-Silveira L, et al. (The Journal of General Virology. 2016; 97:1865-76). FIG. 3 shows the production of a recombinant adeno-SARS-CoV-2 polyprotein in HEK 293T cell.

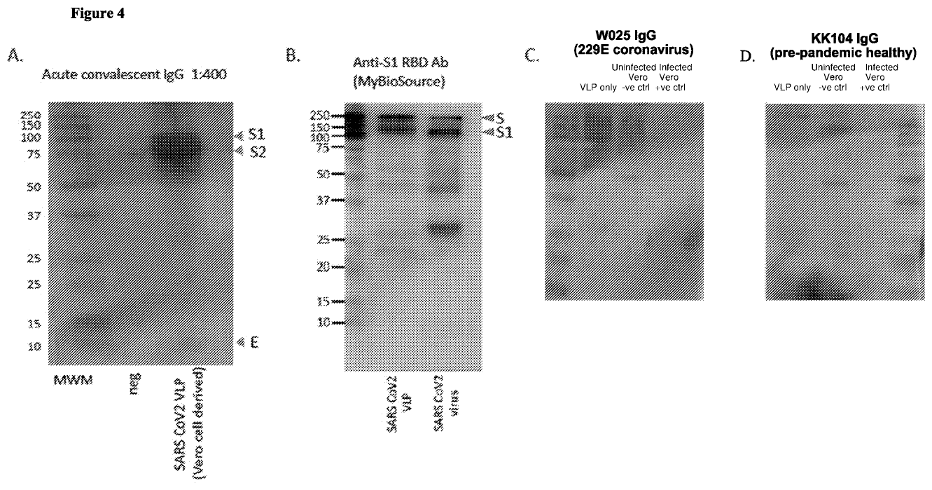

For initial small-scale production, SARS-CoV-2-SEM VLP were produced in Vero cells in 70 cm² tissue culture flasks (Corning Inc, Life Sciences) following infection with the rAd-SARS-CoV-2-SEM virus at a MOI of 1.0. SARS-CoV-2-SEM VLP were then purified from culture supernatants by ultracentrifugation through a 20% sucrose cushion. As shown in FIG. 4, SARS-CoV-2 proteins were detected in the culture supernatant, indicative of the formation of SARS-CoV-2 VLP.

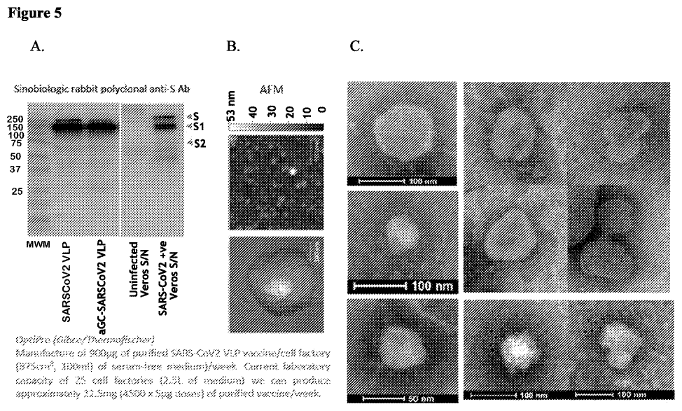

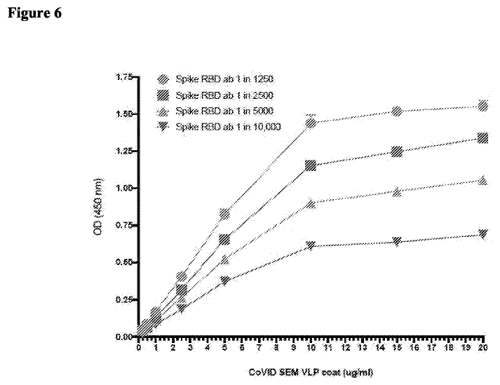

The formation of SARS-CoV-2 VLP was confirmed by atomic force microscopy and transmission electron microscope (TEM), as shown in FIG. 5. The presence of spike proteins on the SARS-CoV-2 VLP was confirmed by electron microscopy and by Western blot analysis (FIGS. 4 and 5). Further analysis showed that the SARS-CoV-2 VLP comprised RBD, as determined by ELISA under native, non-denaturing conditions. This is highlighted by the data in FIG. 6, in which anti-SARS-CoV-2 RBD antibodies (MyBioSource) were used to confirm the presence of the RBD on SARS-CoV-2 VLP coated onto the ELISA plates. The data show that the anti-RBD antibody recognizes the RBD present on native, non-denatured SARS-CoV-2 VLP.

For large-scale production of SARS-CoV-2-SEM VLP, Vero cell factories may suitably be infected with rAd-SARS-CoV-2-SEM at an MOI of 1.0 added directly to culture medium. Cell factory culture supernatants can then be collected 96 hr post-infection and SARS-CoV-2-SEM VLP purified by ultracentrifugation through a sucrose cushion. In is anticipated that this will result in the production of approximately 8 mg of purified SARSCoV2-SEM VLP. With our current laboratory scale production of VLP, we can manufacture up to 1200 μg of purified SARS-CoV-2 VLP vaccine/cell factory (875 cm², 100 ml) of serum-free medium)/week. With our current laboratory capacity of 25 cell factories (2.5 L of medium) we can produce approximately 22.5 mg (4500×5 μg doses) of purified vaccine/week.

### Example 2—Immunization of Mice

To determine the immunogenicity of the SARS-CoV-2 VLP generated by the self-cleaving SARS-CoV-2 VLP constructs, C57BL/6 mice were immunised with two doses of SARS-CoV-2 VLP recovered from Vero cells, as described in Example 1 above. The VLP were administered intramuscularly (IM), 2 weeks apart. Mice were bled by tail vein bleed immediately before the 2^{nd} (booster) dose. One week after the booster, mice were killed and blood, spleen, lymph nodes and liver harvested for analyses.

Anti-SARS-CoV-2 antibody titres were determined by ELISA using SARS-CoV-2 VLP as the coating antigen. Briefly, 96-well flexible, flat-bottomed polyvinyl chloride (PVC) microtiter plates (Nunc) were coated with 50 μl of 5 μg/ml of SARS-CoV-2 VLP per well in Carbonate Coating buffer (100 mM Na₂CO₃ and NaHCO₃). Plates were incubated at 4° C. overnight in a humidified chamber. On the second day, the coating solution was discarded, and blocked with 10% BSA (Bovine Serum Albumin) in PBS (Phosphate Buffered Saline) for at least 2 hours at room temperature in a humidified chamber. The plates were then washed 4 times with PBS and blotted dry. 50 μl of anti-SARS-CoV-2 antibodies in 5% BSA/PBS at 100 μg/ml was added to each well. The plates were incubated at 4° C. overnight in a humidified chamber. On Day 3, the plates were washed four times with PBS and blotted dry. 50 μl/well of anti-human antibody conjugated to horse radish peroxidase (HRP) (Dako, cat #P0161) diluted 1:500 in 5% BSA/PBS was added to each well. The plates were incubated for 1-2 hours at room temperature in a humidified chamber. The plates were washed four times in PBS. TMB (tetramethylbenzidine) substrate is tested with conjugate prior to use by mixing 2 drops of both reagents in a counting chamber and observing a colour change. 50 μl of substrate was added to each well and incubated for 10-15 mins at room temperature to observe a colour change. The colourmetric reaction was terminated by adding 50 μl/well of 0.16 M H₂SO₄. The plate is determined for absorbance on a plate reader using a wavelength of 450 nm.

SARS-CoV-2 neutralising antibody responses can also be determined by an in vitro neutralisation assay using immune sera from mice and the SARS-CoV-2 virus (from Prof Damien Purcell; DMI, PDI).

NKT and CD4+ and CD8+ T cell responses can be determined in lymphocytes isolated from spleens, lymph nodes and livers of vaccinated mice, using methods that will be familiar to persons skilled in the art.

To determine the frequency of SARS-CoV-2-specific memory CD8+ T cells in the spleen of immunized mice, two approaches can be applied: (1) Elispot to measure the frequency of CD8+ T cells that respond to VLP mixed with antigen presenting cells and the target proteins and peptides derived from these VLP; and (2) flow cytometric analysis to determine the memory cell phenotype (central memory (Tcm), effector memory (Tem)) following immunization.

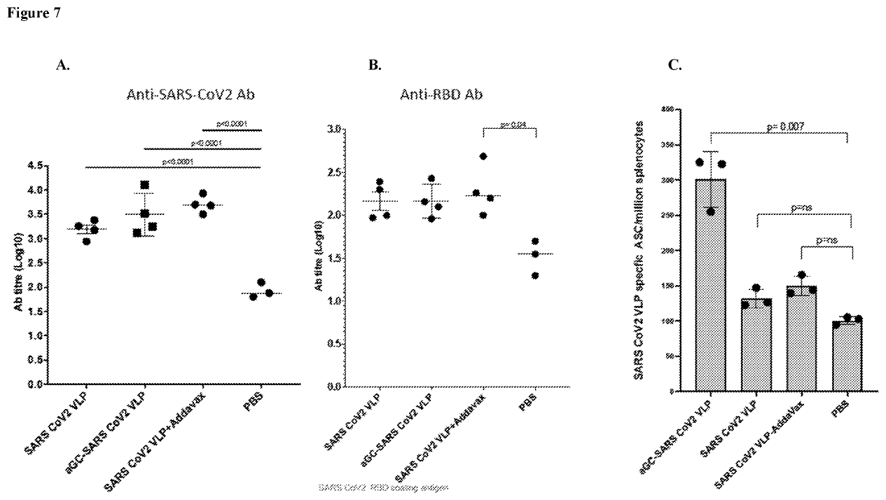

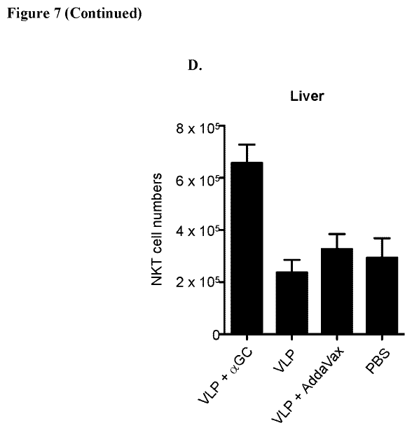

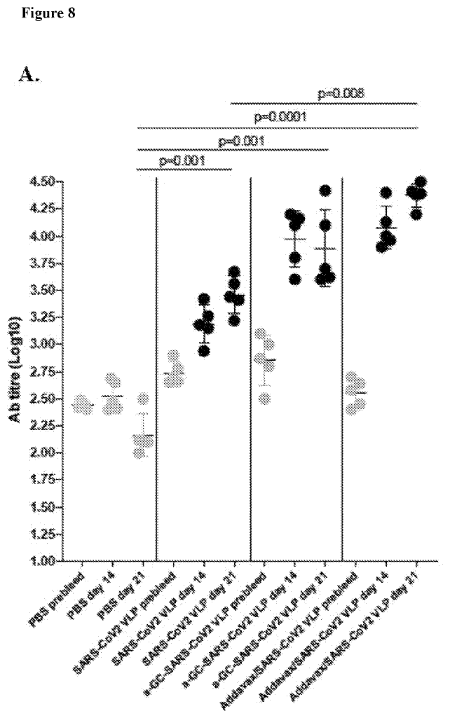

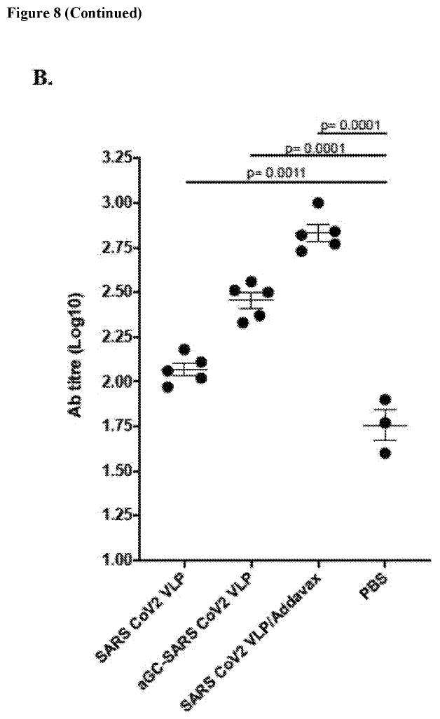

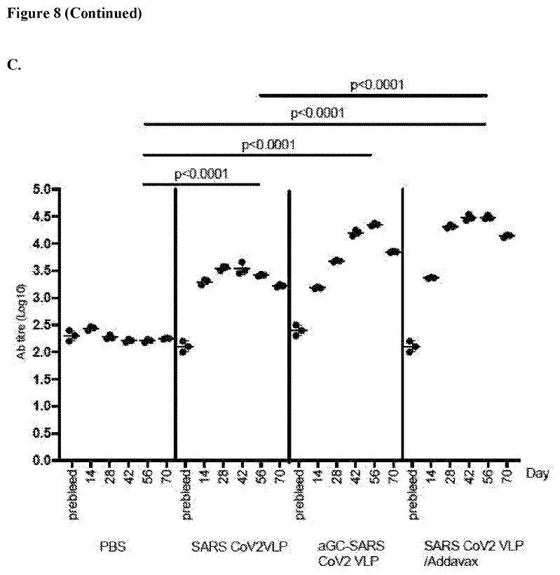

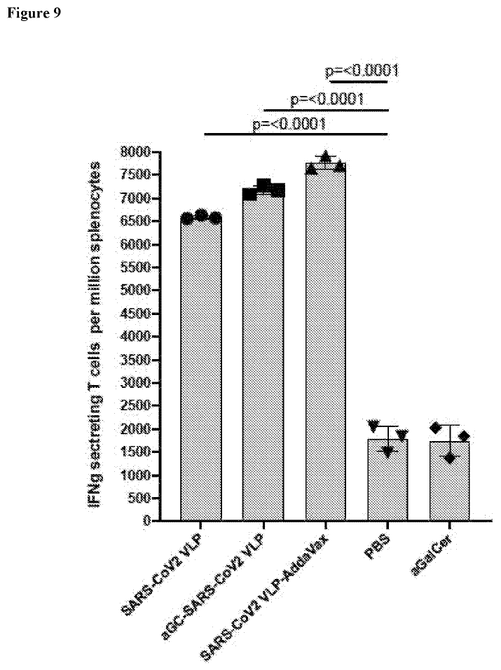

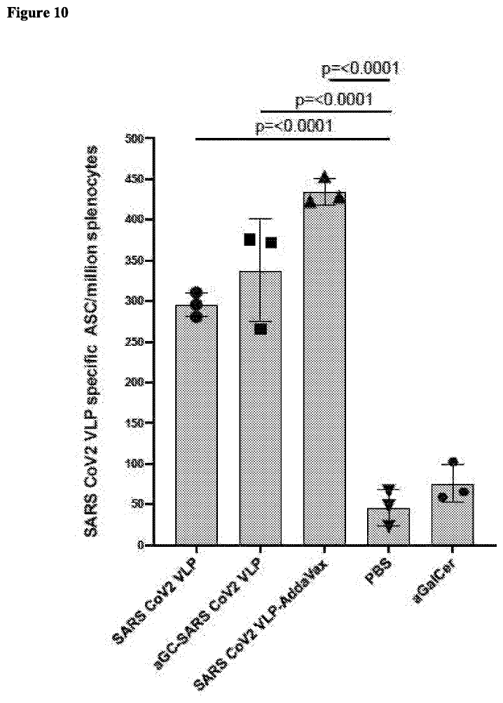

ELISA and ELISPOT analyses demonstrate that administration of SARS-CoV-2 VLP in mice resulted in an elevation of anti-S1 and anti-RBD antibody titres (FIGS. 9A-C and 10A-C), when compared to control (PBS alone). It was also noted that co-administration of SARS-CoV-2 VLP with the lipid adjuvant, α-galactosylceramide (α-GC), resulted in a significant increase in B cell and NKT cell numbers, as well as an increase in the anti-S1 and anti-RBD antibody titres when compared to the administration of SARS-CoV-2 VLP alone. The co-administration of SARS-CoV-2 VLP with the squalene-based, oil-in-water nano-emulsion vaccine adjuvant, AddaVax, had a greater effect on anti-SARS-CoV-2 VLP and RBD antibody, B cell and T cell numbers (FIGS. 7-10).

These data show that a nucleic acid construct encoding a self-cleaving polyprotein comprising structural proteins of SARS-CoV-2, when expressed in a mammalian host cell, undergoes host-cell dependent cleavage to liberate the structural proteins that subsequently self-assemble to form a SARS-CoV-2 VLP. Advantageously, the SARS-CoV-2 VLP were stable enough to generate a humoral and cellular immune response when administered to an immunocompetent subject in vivo, including a robust antibody response with antibodies against the SARS-CoV-2 spike protein. The humoral and cellular immune responses were further enhanced when the SARS-CoV-2 VLP was co-administered with an immunomodulatory agent, such as α-galactosylceramide (α-GC) and AddaVax.

### Example 3—Immunization of Mice—II

A nucleic acid construct was developed to produce a self-cleaving polyprotein comprising the spike (S), membrane (M) and envelope (E) proteins of the B.1.1.7 SARS-CoV-2 isolate (BetaCoV; also referred to herein as the B.1.1.7 variant), the entire contents of which is incorporate herein by reference). A signalase sequence (SEQ ID NO:17) was introduced (i) between the S2 and E; and (ii) between the E and M proteins of the polyprotein to facilitate self-cleavage of the expressed polyprotein and thereby liberate the S1, S2, E and M proteins to allow said proteins to self-assemble and form a SARS-CoV-2 VLP.

To produce the SARS-CoV-2 nucleic acid construct, the genome sequence of the SARS-CoV-2 spike (S), envelope (E) and membrane (M) proteins were derived from GeneBank database. The SARS-CoV-2 nucleic acid construct was synthesized by GeneArt and subcloned into an AdEasy expression system. The SARS-CoV-2 vaccine construct was then cloned into pAdTrack CMV to create the pAdTrack-CMV-SARS-CoV-2. This plasmid was enzymatically digested with PmeI and transformed into AdEasier™ cells by electroporation (Bio-Rad Gene Pulser). Positive clones were confirmed by restriction digestion with PacI, then transformed into Top 10 F′ cells.

rAd-SARS-CoV-2-SEM viruses were produced by transfection of the generated plasmid DNA into 293T cells and high titres of recombinant adenoviruses encoding the structural proteins (rAd-SARS-CoV-2-S/E/M) were produced in 293T cells by serial passaging. Multiplicity of infection (MOI) was determined as described in Earnest-Silveira L, et al. (ibid).

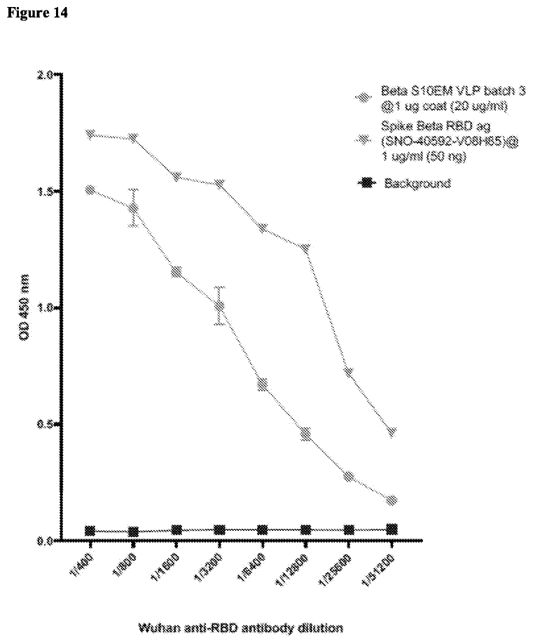

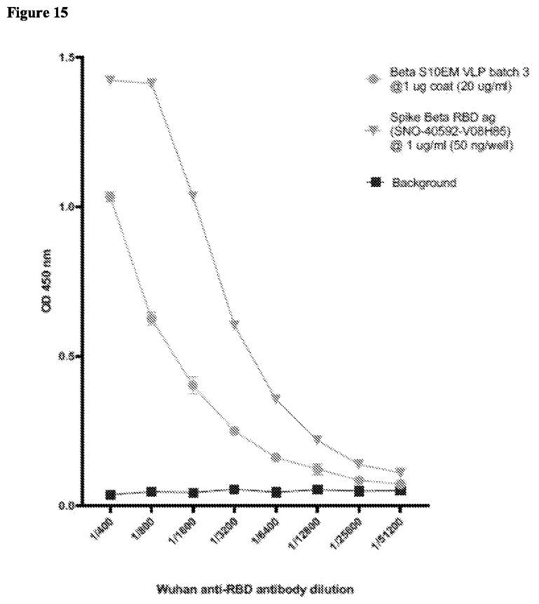

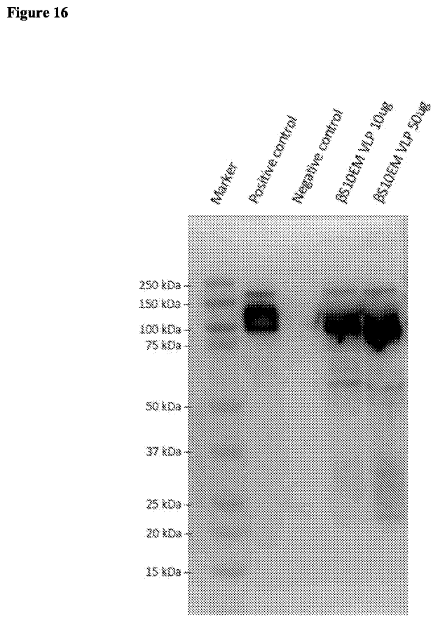

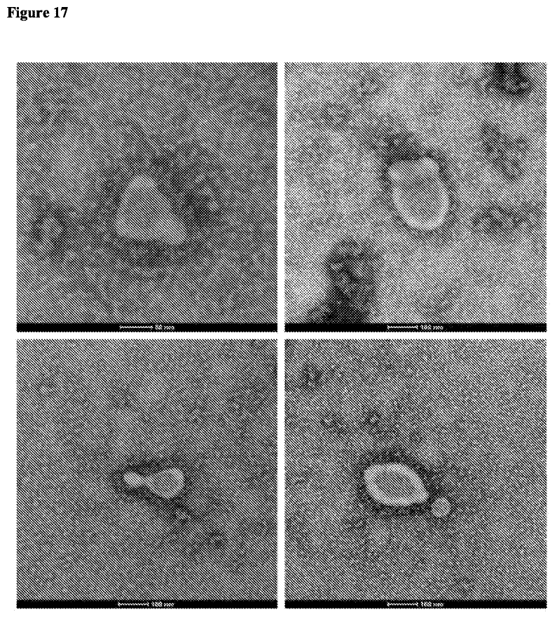

For initial small-scale production, the SARS-CoV-2-SEM VLP were produced in Vero cells in 70 cm² tissue culture flasks (Corning Inc, Life Sciences) following infection with the rAd-SARS-CoV-2-SEM virus at a MOI of 1.0. The SARS-CoV-2-SEM VLP were then purified from culture supernatants by ultracentrifugation through a 20% sucrose cushion. The formation of SARS-CoV-2 VLP by Vero cells was confirmed by transmission electron microscope (TEM), as shown in FIG. 17. Further analysis showed that the SARS-CoV-2 VLP comprised RBD, as determined by ELISA under native, non-denaturing conditions. As shown in FIGS. 14 and 15, the use of anti-SARS-CoV-2 RBD antibodies (MyBioSource) confirmed the presence of the RBD on the SARS-CoV-2 VLP. The presence of spike (S), membrane (M) and envelope (E) proteins on the SARS-CoV-2 VLP was also confirmed by Western blot analysis (see FIG. 16).

To determine the immunogenicity of the SARS-CoV-2 VLP generated by Vero cells transduced to express the self-cleaving SARS-CoV-2 VLP vaccine constructs, C57BL/6 mice were immunised with two doses of the SARS-CoV-2 VLP recovered from Vero cells, as outlined above. The VLP were administered IM, 2 weeks apart. Control animals received phosphate buffered saline by intramuscular injection at the same time points. All mice were bled by tail vein bleed on day 14; for mice immunised with the SARS-CoV-2 VLP, tail vein blood was collected immediately before the 2^{nd} (booster) dose. At 2, 4 and 6 weeks after the booster, mice were killed and blood, spleen, lymph nodes and liver harvested for analyses. In a separate cohort, immunized mice were challenged at week 6 following the booster with SARS-CoV2 and assessed for the number of infectious SARS-CoV-2 virus particles in their lungs.

Anti-SARS-CoV-2 antibody titres were determined by ELISA using SARS-CoV-2 VLP as the coating antigen, as described in Example 2 above.

SARS-CoV-2 neutralising antibody responses were determined by an in vitro neutralisation assay using immune sera from mice and the SARS-CoV-2 virus (from Prof Damien Purcell; DMI, PDI), in accordance with the protocol described in Tan et al. 2020, Nat Biotechnol, 38(9):1073-1078, the entire contents of which is incorporated herein by reference.

The proportion of IFNγ-secreting T cells in the splenocytes of the mice was determined by an Elispot assay.

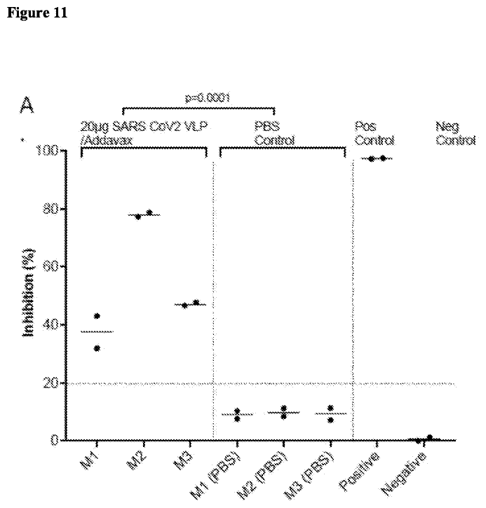

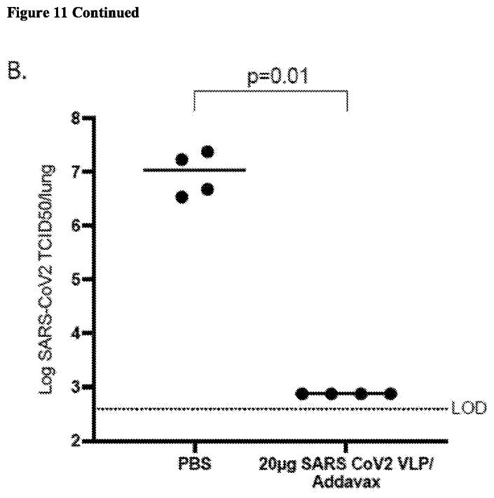

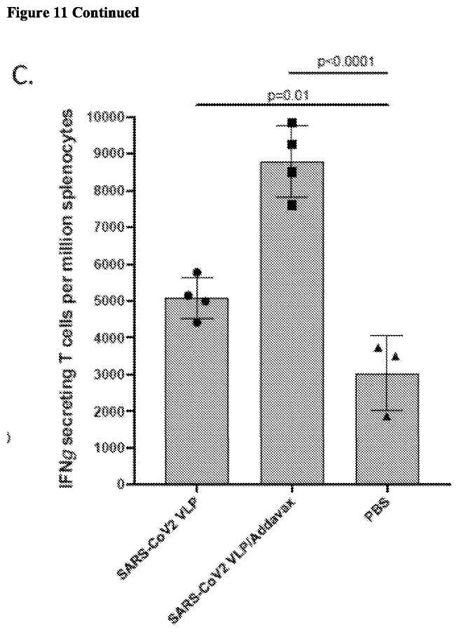

As shown in FIG. 11A, mice immunized with two doses of the SARS-CoV-2 VLP developed neutralizing antibodies, as determined by sVNT assay, when compared to PBS controls (P<0.0001). In mice who received the SARS-CoV-2 VLP, the TCID₅₀ of virus from lungs showed 100% protection against challenge with mouse adapted SARS-CoV-2 virus (see FIG. 11B). These outcomes were associated with an increase in the number of IFNγ-secreting T cells harvested from the spleens of immunized animals when compared to those derived from the spleens of PBS control animals (see FIG. 11C).

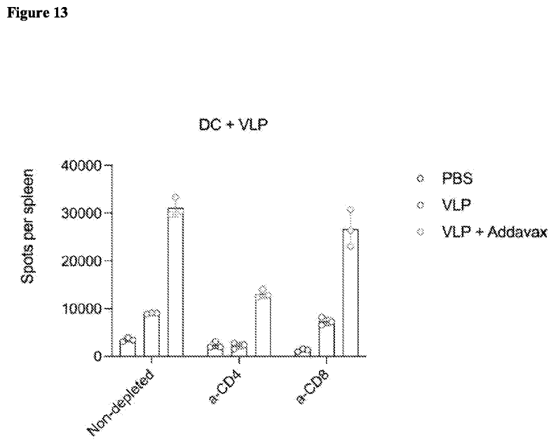

Immunization of mice with the SARS-CoV-2 VLP also produced a predominant CD4−T cell response, as determined by Elispot assays. As shown in FIG. 13, depletion of CD4+ T cells resulted in a significant reduction in the number of spots in both the VLP alone and VLP/Addavax™ vaccinated mice.

### Example 4—Inhibition of SARS-CoV-2 Infection of Human Nasal Epithelial Cells

To determine the extent to which neutralizing anti-SARS-CoV-2 antibodies generated in the immunised mice can inhibit SARS-CoV-2 infection of nasal epithelial cell, an air-liquid-interface (ALI) differentiated human nasal epithelium (HNE) culture system was used as a model of authentic SARS-CoV-2. This model was previously described by Tran et al. (2022; Int. J. Mol. Sci.; 23:835), the entire contents of which is incorporated herein by reference. Briefly, Progenitor cells (basal cells) were isolated from nasal turbinate brushings, expanded under conditionally reprogrammed cell (CRC) culture conditions and differentiated at air-liquid-interface. Differentiated cells were then inoculated with the B.1.1.7 SARSCoV-2 isolate (BetaCoV/; also referred to herein as the B.1.1.7 variant). Infectious virus release into apical washes was then determined by TCID₅₀, while infected cells were visualized by immunofluorescence and confocal microscopy.

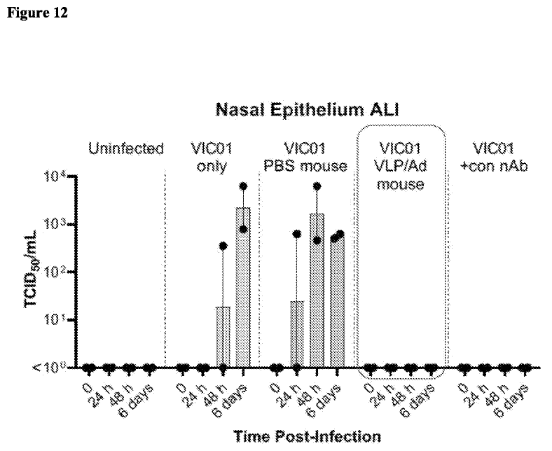

As shown in FIG. 12, the sera of mice immunized with SARS-CoV-2 VLP, co-administered with AddaVax™ (a squalene-based oil-in-water nano-emulsion; Invitrogen) inhibited SARS-CoV-2_{VIC01} infection of human nasal epithelial cells, when compared to sera from PBS control mice or in the presence of SARS-CoV-2_{VIC01} alone.

These data show that SARS-CoV-2 VLP generated by a nucleic acid construct encoding a self-cleaving polyprotein comprising structural proteins of SARS-CoV-2, including the beta spike protein, when employed in a vaccine composition, are capable of raising neutralizing anti-SARS-CoV-2 antibodies that can protect against subsequent SARS-CoV-2 infection.

### Example 5—Dual Constructs Enhance the Production of SARS-CoV-2 VLP

This study was undertaken to compare the use of (i) a single construct encoding an immunogen (the VIC01 SARS-CoV-2 spike protein) and a self-cleaving polyprotein comprising the virus structural proteins and (i) two constructs—a first construct encoding the immunogen and a second construct encoding a self-cleaving polyprotein comprising virus structural proteins, where the virus structural proteins, once liberated from the polyprotein by host cell-dependent peptidases, are capable of self-assembling to form a VLP with the immunogen.

Briefly, a single construct (Mono scβSEM) was prepared comprising a nucleic acid sequence encoding a polyprotein comprising (i) the spike protein of the B.1.1.7 SARSCoV-2 isolate (BetaCoV), (ii) the membrane (M) protein of the B.1.1.7 SARSCoV-2 isolate and (iii) the envelope (E) protein of the B.1.1.7 SARSCoV-2 isolate. In the beta SIOEM SARS-CoV-2 construct, the spike, membrane and envelope proteins of the polyprotein were each separated by the signal peptidase (signalase) sequence of SEQ ID NO:17.

For comparison, dual constructs (Dual βS10scEM) were prepared, with a first construct comprising a nucleic acid sequence encoding the spike protein of the B.1.1.7 SARSCoV-2 isolate (BetaCoV) and a second construct comprising a nucleic acid sequence encoding a polyprotein comprising (i) the membrane (M) protein of the B.1.1.7 SARSCoV-2 isolate and (ii) the envelope (E) protein of the B.1.1.7 SARSCoV-2 isolate, where the membrane and envelope proteins of the polyprotein were each separated by the signal peptidase (signalase) sequence of SEQ ID NO:17.

VLP were produced in Vero cells from the Mono scβSEM and Dual βS10scEM constructs in accordance with the methods set out in Examples 1 and 3, above.

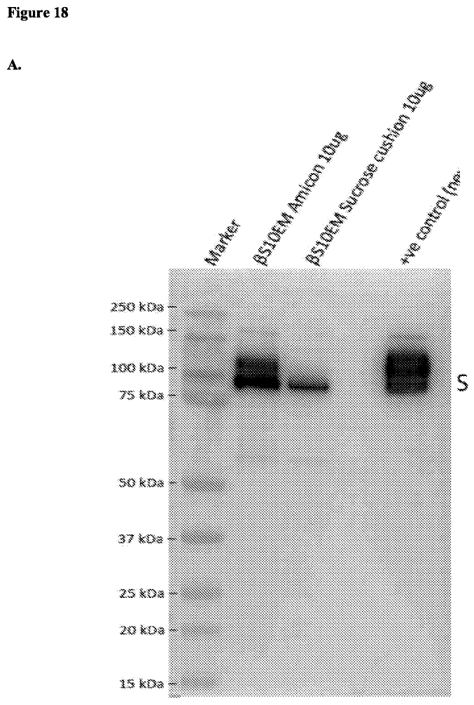

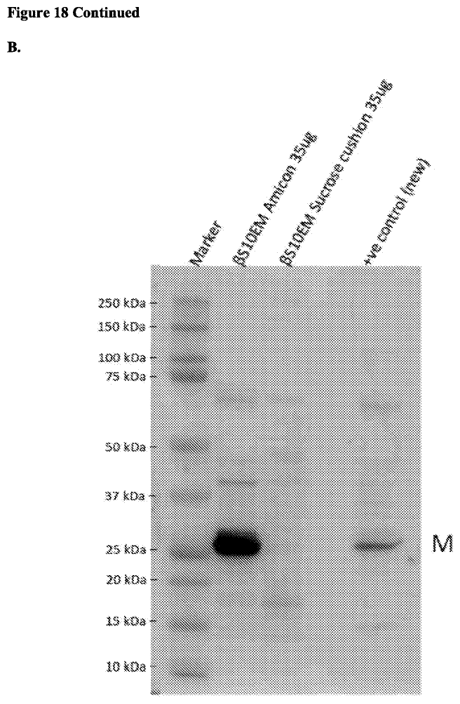

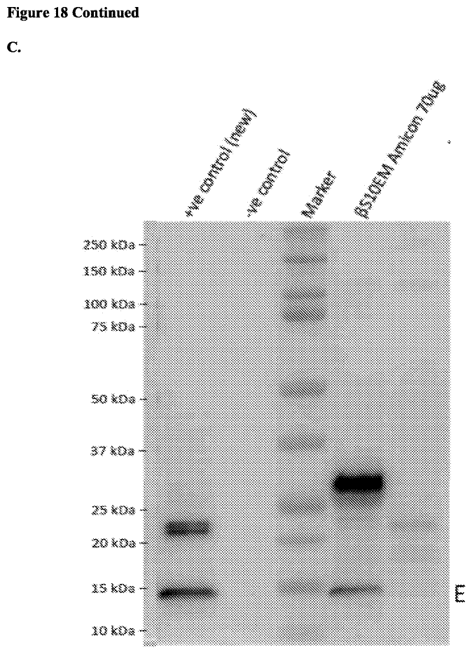

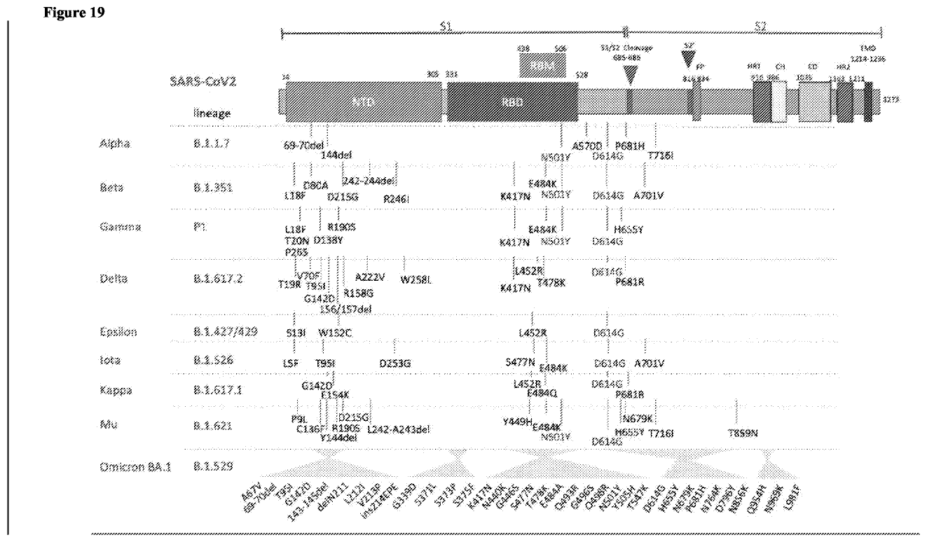

The presence of the spike (S), membrane (M) and envelope (E) proteins on the SARS-CoV-2 VLP produced by the Dual βS10scEM constructs was confirmed by Western blot analysis (see FIGS. 18A-C).

The Dual βS10scEM constructs, when expressed in Vero cells, produced a total of 3.7 mg of SARS-CoV-2 VLP in 500 ml of supernatant. By comparison, the Mono scβSEM construct, when expressed in Vero cells, produced a total of 1.5 mg of SARS-CoV-2 VLP in 500 ml of supernatant. These data show that the Dual βS10scEM constructs unexpectedly gave rise to a much greater yield of VLP than the Mono scβSEM construct.

Throughout the specification, the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A nucleic acid construct for producing a virus-like particle (VLP) capable of raising an immune response against severe acute respiratory syndrome coronavirus (SARS-CoV), wherein the construct comprises a nucleic acid sequence encoding a polyprotein, wherein the polyprotein comprises (i) an immunogen; and (ii) two or more viral structural proteins, wherein the immunogen comprises a B cell epitope and/or a T cell epitope of a SARS-CoV surface protein and wherein at least two of the two or more viral structural proteins are separated by a signal peptidase sequence such that, when the polyprotein is expressed in a host cell, the signal peptidase sequence undergoes host cell peptidase-dependent cleavage to liberate the two or more viral structural proteins, thereby allowing the liberated structural proteins to self-assemble into a VLP carrying the immunogen.

2. The vaccine construct according to claim 1, wherein each of the two or more viral structural proteins are separated by a signal peptidase sequence.

3. The nucleic acid construct according to claim 1, wherein the SARS-CoV is a Severe acute respiratory syndrome coronavirus 2 (SARS-CoV-2).

4.-5. (canceled)

6. The nucleic acid construct according to claim 1, wherein the two or more viral structural proteins are selected from the group consisting of:
(i) Subunit 1 of SARS-CoV-2 spike protein (S1) comprising an amino acid sequence of SEQ ID NO:3 or an amino acid having at least 85% sequence identity thereto;
(ii) Subunit 2 of SARS-CoV-2 spike protein (S2) comprising an amino acid sequence of SEQ ID NO:4 or an amino acid having at least 85% sequence identity thereto;
(iii) SARS-CoV-2 membrane protein comprising an amino acid sequence of SEQ ID NO:6 or an amino acid having at least 85% sequence identity thereto;
(iv) SARS-CoV-2 envelope protein comprising an amino acid sequence of SEQ ID NO:7 or an amino acid having at least 85% sequence identity thereto; and
(v) a SARS-CoV-2 nucleoprotein comprising an amino acid sequence of SEQ ID NO:8 or an amino acid having at least 85% sequence identity thereto.

7. The nucleic acid construct according to claim 6, wherein the polyprotein comprises:
(vi) Subunit 1 of SARS-CoV-2 spike protein (S1) comprising an amino acid sequence of SEQ ID NO:3 or an amino acid having at least 85% sequence identity thereto;
(vii) Subunit 2 of SARS-CoV-2 spike protein (S2) comprising an amino acid sequence of SEQ ID NO:4 or an amino acid having at least 85% sequence identity thereto;
(viii) SARS-CoV-2 membrane protein comprising an amino acid sequence of SEQ ID NO:6 or an amino acid having at least 85% sequence identity thereto;
(ix) SARS-CoV-2 envelope protein comprising an amino acid sequence of SEQ ID NO:7 or an amino acid having at least 85% sequence identity thereto; and
(x) a SARS-CoV-2 nucleoprotein comprising an amino acid sequence of SEQ ID NO:8 or an amino acid having at least 85% sequence identity thereto.

8. The nucleic acid construct according to claim 1, wherein the immunogen comprises a B cell epitope and/or a T cell epitope of a SARS-CoV surface protein, or a mimotope thereof.

9. The nucleic acid construct according to claim 8, wherein the SARS-CoV surface protein is selected from the group consisting of:
(i) Subunit 1 of SARS-CoV-2 spike protein (S1) comprising an amino acid sequence of SEQ ID NO:3 or an amino acid having at least 85% sequence identity thereto; and
(ii) Subunit 2 of SARS-CoV-2 spike protein (S2) comprising an amino acid sequence of SEQ ID NO:4 or an amino acid having at least 85% sequence identity thereto.

10. The nucleic acid construct according to claim 1, wherein the immunogen comprises a B cell epitope and/or a T cell epitope of a receptor binding domain of SARS-CoV.

11. The nucleic acid construct according to claim 10, wherein the receptor binding domain of SARS-CoV comprises an amino acid sequence of SEQ ID NO:5, or an amino acid having at least 85% sequence identity thereto.

12. The nucleic acid construct according to claim 1, herein the polyproteins comprises a modified SARS-CoV-2 nucleoprotein, wherein the modified SARS-CoV-2 nucleoprotein is unable to package RNA.

13. (canceled)

14. The nucleic acid construct according to claim 1, wherein signal peptidase sequence is a signal peptidase sequence utilized by a hepatitis C virus, or a cleavable variant thereof.

15. The nucleic acid construct according to claim 14, wherein the signal peptidase sequence comprises an amino acid sequence selected from the group consisting of SEQ ID Nos:17-24, and amino acid sequences having at least 80% sequence identify to any of the foregoing.

16.-17. (canceled)

18. A method of producing a VLP, the method comprising:
(i) introducing the nucleic acid construct according to claim 1 into a host cell; and
(ii) culturing the host cell of (i) under conditions and for a period of time sufficient for the host cell or produce the VLP.

19. (canceled)

20. A system or composition for producing a virus-like particle (VLP) capable of raising an immune response against severe acute respiratory syndrome coronavirus (SARS-CoV), the system or composition comprising (i) a first construct comprising a nucleic acid sequence encoding an immunogen, wherein the immunogen comprises a B cell epitope and/or a T cell epitope of a SARS-CoV surface protein; and (ii) a second construct comprising a nucleic acid sequence encoding a polyprotein, wherein the polyprotein comprises two or more viral structural proteins, wherein at least two of the two or more viral structural proteins are separated by a signal peptidase sequence such that, when the polyprotein and the immunogen are expressed in a host cell, the signal peptidase sequence of the polyprotein undergoes host cell peptidase-dependent cleavage to liberate the two or more viral structural proteins, thereby allowing the liberated structural proteins and the immunogen to self-assemble into a VLP.

21.-24. (canceled)

25. A VLP produced by the method according to claim 18.

26. A vaccine composition comprising the nucleic acid construct according to claim 1.

27. The composition according to claim 26, further comprising an adjuvant.

28. (canceled)

29. The composition according to claim 26, wherein the adjuvant is an immunostimulatory lipid.

30. The composition according to claim 29, wherein the immunostimulatory lipid is a glycolipid.

31.-32. (canceled)

33. The composition according to claim 30, wherein the glycolipid is α-galactosylceramide or α-glucosylceramide.

34. (canceled)

35. A method of raising an immune response to an immunogen, the method comprising administering to a subject in need thereof the nucleic acid construct according to claim 1.

36.-37. (canceled)

38. A kit comprising the nucleic acid construct according to claim 1.

39. A host cell comprising the nucleic acid construct according to claim 1.

40. (canceled)
